(19) United States
(12) Patent Application Publication
(10) Pub. No.: US 2023/0031629 A1
(43) Pub. Date: Feb. 2, 2023

(54) ANTIGEN LOADING

(71) Applicant: DendroCyte BioTech Pty Ltd., Balmain, New South Wales (AU)

(72) Inventors: Georgina Jane CLARK, Balmain, New South Wales (AU); Sarah Isabella Mackenzie LINDQUIST, Drummoyne, New South Wales (AU)

(21) Appl. No.: 17/782,263

(22) PCT Filed: Dec. 4, 2020
(86) PCT No.: PCT/AU2020/051329

§ 371 Date: Jun. 3, 2022

(30) Foreign Application Priority Data
Dec. 5, 2019 (AU) AU2019904614

(51) Int. Cl. C07K 16/28; A61K 35/17; A61K 35/15; A61K 39/00; A61K 39/385; A61P 31/12; A61P 35/00; A61P 37/04; A61K 39/12; A61K 47/68

(52) U.S. Cl. CPC C07K 16/2803; A61K 35/17; A61K 35/15; A61K 39/0011; A61K 39/385; A61P 31/12; A61P 35/00; A61P 37/04; A61K 39/12; A61K 47/6801; A61K 2039/505; A61K 2039/5154; C07K 2317/24; C07K 2317/77; C07K 2319/00; C07K 2317/565

(57) ABSTRACT

The disclosure relates to a method of antigen loading an antigen presenting cell or precursor thereof with a target antigen for presentation of the target antigen to a T cell, comprising contacting the antigen presenting cell or precursor thereof with a CD300f binding protein in the presence of the target antigen The disclosure also relates to compositions for antigen loading antigen presenting cells or precursors thereof, to immunoconjugates for antigen loading antigen presenting cells or precursors thereof, and use of antigen-loaded antigen presenting cells and immunoconjugates for promoting or increasing a T cell response to a target antigen in a subject.

The present application claims priority from Australian provisional application no. 2019904614 filed 5 Dec. 2019, the entirety of which is incorporated herein by reference.

### FIELD

The present invention relates to a method for antigen loading an antigen presenting cell or a precursor thereof, to use of the antigen-loaded antigen presenting cell or a precursor thereof for promoting or increasing a T cell response to a target antigen, to compositions for antigen loading antigen presenting cells or a precursor thereof, and to a CD300f binding protein immunoconjugate for antigen loading antigen presenting cells or a precursor thereof, or for promoting or increasing an immune response to a target antigen in a subject.

### BACKGROUND

Immunotherapy is a form of treatment that is designed to activate or suppress the immune system to treat a condition or disease. Immunotherapy has become of particular interest in the treatment of cancer.

Cell-based immunotherapies are effective for some cancers. Immune effector cells such as T lymphocytes (e.g., CD4+ helper T cells and cytotoxic CD8+ T cells), can be targeted to specific target antigens, such as cancer antigens, if the target antigen is presented to the effector cells on the surface of antigen presenting cells. Presentation of the target antigen on the surface of antigen presenting cells to T cells initiates an antigen specific T cell response against cells expressing the target antigen.

Dendritic cells (DCs) are antigen presenting cells for the induction of antigen specific T cell responses. Recent approaches in cell-based immunotherapy involves producing dendritic cell vaccines by loading antigen presenting cells such as dendritic cells with a target antigen. Loading of target antigen into the dendritic cell is usually achieved by isolating dendritic cells from the subject and introducing the target antigen into the dendritic cell. The target antigen is then processed by the dendritic cell and presented on the surface of the dendritic cell, typically complexed with MHC I, to produce a dendritic cell vaccine (DC vaccine). The dendritic cell vaccine can then be administered to the subject to induce an immune response to the target antigen.

Despite recent advances in immunotherapy to treat cancer, DC vaccination therapy has yet to become widely used. DC vaccination has been shown to be safe and effective but cost and deliverability of current ex vivo loading strategies makes access difficult.

What is needed is alternative approaches for antigen loading antigen presenting cells, for DC vaccination and other therapies.

### SUMMARY

CD300f is a member of the CD300 family of immunoregulatory molecules encoded by a gene complex on chromosome 17q25. It is a transmembrane glycoprotein with a cytoplasmic region and an extracellular domain. The cytoplasmic region contains both inhibitory ITIMs and PI3K phosphorylation sites. There are a number of isoforms of the CD300f molecule, but they all share the same Ig-like extracellular domain.

The inventors have found that CD300f is expressed on antigen presenting cells and precursors thereof, is able to be internalized, and has an expression profile that is more restricted than that of DEC-205. The inventors have found that CD300f monoclonal antibodies are able to deliver a target antigen and induce a target antigen specific T cell response in vitro. The inventors have also shown that an anti-CD300f antibody activates dendritic cells without adjuvant and promotes migration of the dendritic cells.

A first aspect provides a method of antigen loading an antigen presenting cell or precursor thereof with a target antigen for presentation of the target antigen to a T cell, the method comprising contacting the antigen presenting cell or precursor thereof with a CD300f binding protein in the presence of the target antigen.

A second aspect provides a method of antigen loading an antigen presenting cell or precursor thereof with a target antigen for presentation of the target antigen to a T cell, and promoting activation of the antigen presenting cell or precursor thereof, the method comprising contacting the antigen presenting cell or precursor thereof with a CD300f binding protein in the presence of the target antigen.

A third aspect provides a method of presenting a target antigen to a T cell, the method comprising:

antigen loading an antigen presenting cell or precursor thereof with a target antigen by contacting the antigen presenting cell or precursor thereof with a CD300f binding protein in the presence of the target antigen to produce an antigen-loaded antigen presenting cell or precursor thereof; and

culturing the antigen-loaded antigen presenting cell or precursor thereof with a population comprising T cells under conditions which permit presentation of the target antigen to the T cells of the population.

A fourth aspect provides a method of promoting or increasing a T cell response to a target antigen, comprising:

antigen loading an antigen presenting cell or precursor thereof with a target antigen by contacting an antigen presenting cell or precursor thereof with a CD300f binding protein in the presence of the target antigen to produce an antigen-loaded antigen presenting cell or precursor thereof; and

culturing the antigen-loaded antigen presenting cell or precursor thereof with a population comprising T cells under conditions which permit presentation of the target antigen to the T cells of the population to stimulate a target antigen specific T cell response.

A fifth aspect provides a method of promoting or increasing a T cell response to a target antigen in a subject, the method comprising administering to the subject an effective amount of a CD300f binding protein and the target antigen.

An alternative fifth aspect provides a CD300f binding protein and a target antigen for use in promoting or increasing a T cell response to the target antigen in a subject; or use of a CD300f binding protein and a target antigen in the manufacture of a medicament for promoting or increasing a T cell response to the target antigen in a subject.

A sixth aspect provides a method of treating a disease or condition requiring a T cell response to a target antigen, the method comprising administering an effective amount of a CD300f binding protein and the target antigen.

An alternative sixth aspect provides a CD300f binding protein and a target antigen for use in treating a disease or condition requiring a T cell response to the target antigen; or use of a CD300f binding protein and a target antigen in the manufacture of a medicament for treating a disease or condition requiring a T cell response to the target antigen.

A seventh aspect provides a composition for antigen loading an antigen presenting cell or precursor thereof with a target antigen, the composition comprising a CD300f binding protein and the target antigen.

An eighth aspect provides a pharmaceutical composition for promoting or increasing a T cell response to a target antigen, the composition comprising a CD300f binding protein and the target antigen.

A ninth aspect provides immunoconjugate for antigen loading an antigen presenting cell or precursor thereof with a target antigen, the immunoconjugate comprising a CD300f binding protein coupled to the target antigen.

A tenth aspect provides a method of producing an antigen-loaded antigen presenting cell or a precursor thereof which is capable of presenting a target antigen to a T cell, the method comprising contacting an antigen presenting cell or precursor thereof with a CD300f binding protein in the presence of the target antigen for sufficient time to permit uptake of the target antigen.

An eleventh aspect provides an antigen-loaded antigen presenting cell or precursor thereof which is capable of presenting a target antigen to a T cell, wherein the antigen-loaded antigen presenting cell or precursor thereof is produced by contacting an antigen presenting cell or precursor thereof with a CD300f binding protein in the presence of the target antigen for sufficient time to permit uptake of the target antigen.

A twelfth aspect provides a method of promoting a T cell response to a target antigen, comprising incubating the antigen-loaded antigen presenting cell or precursor thereof of the eleventh aspect with a population comprising T cells under conditions which promote presentation of the target antigen to the T cells.

A thirteenth aspect provides a method of promoting or increasing a T cell response to a target antigen in a subject, the method comprising administering to the subject an effective amount of the antigen-loaded antigen presenting cell or precursor thereof of the eleventh aspect.

An alternative thirteenth aspect provides an antigen-loaded antigen presenting cell or precursor thereof of the eleventh aspect for use in promoting or increasing a T cell response to a target antigen in a subject; or use of an antigen-loaded antigen presenting cell or precursor thereof of the eleventh aspect in the manufacture of a medicament for promoting or increasing a T cell response to a target antigen in a subject.

A fourteenth aspect provides a method of treating a disease or condition requiring a T cell response to a target antigen, the method comprising administering an effective amount of the antigen-loaded antigen presenting cell or precursor thereof of the eleventh aspect.

An alternative fourteenth aspect provides an antigen-loaded antigen presenting cell or precursor thereof of the eleventh aspect for use in treating a disease or condition requiring a T cell response to a target antigen; or use of an antigen-loaded antigen presenting cell or precursor thereof of the eleventh aspect in the manufacture of a medicament for treating a disease or condition requiring a T cell response to a target antigen.

A fifteenth aspect provides a method of promoting or increasing an immune response to a target antigen in a subject, the method comprising administering to the subject an effective amount of the antigen-loaded antigen presenting cell or precursor thereof of the eleventh aspect.

An alternative fifteenth aspect provides an antigen-loaded antigen presenting cell or precursor thereof of the eleventh aspect for use in promoting or increasing an immune response to a target antigen in a subject; or use of an antigen-loaded antigen presenting cell or precursor thereof of the eleventh aspect in the manufacture of a medicament for promoting or increasing an immune response to a target antigen in a subject.

A sixteenth aspect provides a method of promoting or increasing a T cell response to a target antigen in a subject, the method comprising administering to the subject an effective amount of the immunoconjugate of the ninth aspect.

An alternative sixteenth aspect provides an immunoconjugate of the ninth aspect for use in promoting or increasing a T cell response to a target antigen in a subject; or use of an immunoconjugate of the ninth aspect in the manufacture of a medicament for promoting or increasing a T cell response to a target antigen in a subject.

A seventeenth aspect provides a method of treating a disease or condition requiring a T cell response to a target antigen, the method comprising administering an effective amount of the immunoconjugate of the ninth aspect.

An alternative seventeenth aspect provides an immunoconjugate of the ninth aspect for use in treating a disease or condition requiring a T cell response to a target antigen; or use of an immunoconjugate of the ninth aspect in the manufacture of a medicament for treating a disease or condition requiring a T cell response to a target antigen.

An eighteenth aspect provides a method of promoting activation of an antigen presenting cell or precursor thereof, comprising contacting the antigen presenting cell or precursor thereof with a CD300f binding protein.

An alternative eighteenth aspect provides a CD300f binding protein for use in promoting activation of an antigen presenting cell or precursor thereof; or use of a CD300f binding protein in the manufacture of a medicament for promoting activation of an antigen presenting cell or precursor thereof.

A nineteenth aspect provides a method of promoting migration of an antigen presenting cell or precursor thereof towards CCL19 and/or CCL21, comprising contacting the antigen presenting cell or precursor thereof with a CD300f binding protein.

A twentieth aspect provides a method of increasing CCR7 expression in an antigen presenting cell or precursor thereof, comprising contacting the antigen presenting cell or precursor thereof with a CD300f binding protein.

A twenty first aspect provides a fusion protein comprising a CD300f binding protein and a target antigen.

A twenty second aspect provides a CD300f binding protein which comprise a heavy chain variable region comprising the amino acid sequence represented by SEQ ID NO: 15, and a light chain variable region comprising the amino acid sequence represented by SEQ ID NO: 16.

A twenty third aspect provides a composition comprising a CD300f binding protein which comprise a heavy chain variable region comprising the amino acid sequence represented by SEQ ID NO: 15, and a light chain variable region comprising the amino acid sequence represented by SEQ ID NO: 16.

A twenty fourth aspect provides a nucleic acid encoding a CD300f binding protein which comprise a heavy chain variable region comprising the amino acid sequence represented by SEQ ID NO: 15, and a light chain variable region comprising the amino acid sequence represented by SEQ ID NO: 16.

### BRIEF DESCRIPTION OF THE DRAWINGS

FIG. 1A is graphs showing internalisation of anti-CD302, anti-CMRF56, anti-CD300f, and anti-DEC205 surface antibodies at 37° C. Internalisation was assessed by comparing total antibody over time (Total, closed symbol) measured using PE labelled MMRI-20 and DCR-2 on HL60, a monocytic cell line, and FITC labelled anti-CMRF-56 and MMRI-7 on KMH2. This was compared with remaining surface Ig detected with goat anti-mouse IgG AF647 (Invitrogen, A21237).

FIG. 1B is graphs showing the effect of activation on CD300f rate of internalisation in monocyte derived DC (MoDC). Internalisation was assessed on monocyte derived DC (MoDC)+/−LPS 100 ng/ml overnight.

FIG. 2 is a graph showing CD300f, CD302 and DEC205 expression on antigen presenting cells. PBMC were isolated from N=3 healthy donors' peripheral blood. DC subsets where determined by flow cytometry analysis using an antibody panel. Expression of CD300f, CD302 and DEC205 was determined by staining on ice for 20 min with biotinylated antibodies against each antigen and then detecting with streptavidin-PE 1:100 (BD 54061) for 20 min on ice.

FIG. 3 is a graph showing CD302, CD300f and DEC205 mRNA expression across normal tissue by RNA sequencing. The data from this analysis was extracted from dbGaP accession number phs0024.vN.pN.

FIG. 4A is a graph showing surface expression of CD300f on human antigen presenting cell subsets (CD11c, CD1cDC, CD141DC, pDC DC, CD16 DC) and monocytes using DCR-2 or CMRF-81 (isotype control) with flow cytometry.

FIG. 4B is graphs showing CD300f expression on myeloid subsets and monocytes from a single donor.

FIG. 5 is a graph showing T Cell response to antibody directed antigen uptake. CD14+ cells were positively selected from PBMC of N=3 healthy donors by staining PBMC with CD14 microbead (Miltenyi Biotec, 130-050-201) for 15 min at 4° C. and magnetic separation using the autoMACS separator. CD14+ cells where differentiated into Mo-DC by incubating CD14+ cells at 3.33×10⁶/mL in complete AB media supplemented with GM-CSF 800 U/mL and IL-4 1000 u/mL for 5 days. CD3 positive cells were isolated from PBMC using EasySep Human T cell Isolation Kit (Stemcell, 17951) and frozen on day 1. Mo-DC were loaded with CMV antigen on day 5 by staining with primary biotinylated antibody for 20 min on ice and then stained with HCMV-pp65 delivery reagent (Miltenyi Biotec 130-095-406) for 10 mins, washed and then incubated overnight with LPS 100 ng/ml. On day 5, autologous T cells were thawed, rested for 2 hours and stained with CSFE before being added to the Mo-DC at a ratio of 1:10 for 5 days at which point cells were harvested and phenotype assessed by flow cytometry. Graph shows percent of T cells divided (percentage of CD3 CSFE low T cells).

FIG. 6 is graphs showing the effect of crosslinking with CD300 family members on activation marker expression on myeloid DC. 96 well flat bottom tissue culture plates (Fal 353072) were coated with 10 ug/ml of DCR-2 (anti-CD300f), UPH2 (anti-CD300e, Biolegend, 339702), control CMRF-81 antibody and CMRF-35 (anti CD300a/c) supernatant overnight at 4° C. then washed with PBS. Myeloid DC isolated using EasySep Human Myeloid DC Enrichment Kit (Stem Cell Technologies, 19061) and 1×10¹ myeloid DC were added in 200 ul of RPMI 10% AB complete media incubated at 37° C. and 5% CO2 for 18 hours. Supernatant was collected for cytokine analysis and cells harvested for phenotype analysis using CD80 Pe-Cy7 (L307.4, BD561135), CD83 FITC (Hb15a, IM2410U), CD86 BV650 (IT2.2, Biolegend) and HLADR APC-H7 (1243, Biolegend) by flow cytometry.

FIG. 7A is graphs showing allogeneic MLR with myeloid DC crosslinked with DCR-2 or CMRF-81. Myeloid DC were crosslinked with DCR-2 or CMRF-81 (as indicated) and mixed in a 1:5 ratio with CSFE labelled T cells and incubated in RMPI 10% AB media for 6 days. At day 6 cells were harvest and analysed by flow cytometry after staining with CD3 AF 700 (SP34-2, BD 55917), CD4 PerCP Cy5.5 (RPA-T4, BD 560650), CD8 BV421 (RPA-T8, BD 562428). Percent of cells divided determine by frequency of CSFE low cells in each population.

FIG. 7B is graphs showing the concentration of cytokines IFN-γ, IL-10 and IL-17a in supernatants of T cells incubated with myeloid DC crosslinked with DCR-2 or CMRF-81. Supernatant was collected at day 6 and cytokine concentration analysed (n=3) using LEGENDplex Human Inflammation Panel Kit (Cat. No. 740409).

FIGS. 8A and 8B are graphs showing that crosslinking through DCR-2 changes surface molecule expression. FIG. 7A is graphs showing surface marker expression of HLA-DR, CD80, PD-L1, CD83, CD86 or TIM-3 on myeloid DC assessed using flow cytometry following isolation of myeloid DC using EasySep Human Myeloid DC Enrichment Kit (Stem Cell Technologies, 19061) and crosslinking with CMRF-81 or DCR-2. To crosslink the myeloid DC, 1×10¹ myeloid DC were added in 200 ul of RPMI 10% AB complete media incubated at 37° C. and 5% CO₂ for 18 hours with 10 ug/ml of DCR-2 or control CMRF-81 antibody. FIG. 8B is graphs showing surface marker expression of HLA-DR, CD80, PD-L1, CD83, CD86 or TIM-3 on monocytes assessed using flow cytometry following isolation of monocytes using magnetic separation and CD14 Microbeads (130-050-201), and crosslinking for 18 hours with CMRF-81 or DCR-2.

FIGS. 9A and 9B are graphs showing the effect of crosslinking with DCR-2 on CCR7 expression and migration. FIG. 9A is graphs showing CCR7 expression and migration to CCL21 and CCL19 of myeloid dendritic cells crosslinked with DCR-2 or CMRI-81. FIG. 9B is graphs showing CCR2 expression and migration to CCL2 of monocytes crosslinked with DCR-2 or CMRI-81. Crosslinked myeloid DC were harvested and stained with CCR7 PE (R&D, FAB197P) for 15 min at 37° C. Migration of crosslinked DC or monocytes was assessed by determining number of DC or monocytes that migrated across transwells (Corning, 3421) towards CCL19 at a concentration of 0.1 ug/ml in 4 hours or CCL21 at 100 ng/ml in 2 hours, or towards CCL2 in 2 hours, all in RPMI (1% BSA, PSG) compared to no chemokine. DC were harvested from the bottom of the transwell stained with Lin2 FITC (CD3/14/19/20/56, BD 643397) and HLA-DR APC-H7 (I243, Biolegend) and resuspended in 200 ul FACS buffer with Count bright Absolute Count Beads 2500/ml. Number of cells migrated was calculated as number of (lin-DR+ cells/counted beads)×5000 beads. Migration index=# cells migrated chemokine/# cells migrated no chemokine. Student T test was applied (*p<0.05).

FIG. 10 is graphs showing the effect of crosslinking DCR-2 on monocytes. 96 well flat bottom tissue culture plates (Fal 353072) were coated with 10 ug/ml of DCR-2 (anti-CD300f), control CMRF-81 antibody or PBS control at 4° C. then washed with PBS. CD14+ cells were positively selected from N=4 healthy donors using CD14+ microbeads (Miltenyi Biotec 130-050-201) and magnetic separation using an AutoMacs. 1×10¹ CD14+ cells were added in 200 μl of RPMI 10% AB complete media incubated at 37° C. and 5% CO2 for 18 hours. Supernatant was collected for cytokine analysis and cells harvested for flow cytometry analysis using CD80 Pe-Cy7 (L307.4, BD561135), CD83 FITC (Hb15a, IM2410U), CD86 BV650 (IT2.2, Biolegend) and HLADR APC-H7 (1243, Biolegend).

FIG. 11 shows the nucleotide sequence of the light chain variable region of DCR-2, and the amino acid sequence of the mouse (above) and humanised (below) light chain variable region of DCR-2. CDR sequences are in boxes. Amino acids that have been substituted to humanise the sequence are underlined in the humanised sequence.

FIG. 12 shows the nucleotide sequence of the heavy chain variable region of DCR-2, and the amino acid sequence of the mouse (above) and humanised (below) light chain variable region of DCR-2. CDR sequences are in boxes. Amino acids that have been substituted to humanise the sequence are underlined in the humanised sequence.

FIG. 13 is graphs showing binding of mouse DCR-2 (mDCR-2), chimeric DCR-2 and humanised DCR-2 to U937 cells.

### DETAILED DESCRIPTION

The present disclosure relates in one form to antigen loading of an antigen presenting cell or precursor thereof with a target antigen for presentation of the target antigen to a T cell.

As used herein, the phrase “antigen loading an antigen presenting cell or precursor thereof with a target antigen” refers to introducing a target antigen into an antigen presenting cell or a precursor thereof such that the target antigen or a portion thereof is presented on the surface of the cell in a manner that is capable of promoting an antigen specific T cell response against the target antigen.

The inventors have found that contacting an antigen presenting cell or precursor thereof with a CD300f binding protein in the presence of a target antigen results in uptake of the binding protein and the target antigen, and presentation of the target antigen on the cell surface of the antigen presenting cell in a manner that is capable of promoting an antigen specific T cell response to the target antigen. The inventors therefore envisage that CD300f binding proteins can be used to prepare, for example, DC vaccines in which a target antigen is loaded into antigen presenting cells or precursors thereof using the CD300f binding protein.

Accordingly, one aspect provides a method for antigen loading an antigen presenting cell or precursor thereof with a target antigen for presentation of the target antigen to a T cell, the method comprising contacting the antigen presenting cell or precursor thereof with a CD300f binding protein in the presence of the target antigen.

Another aspect provides a method of producing an antigen-loaded antigen presenting cell or precursor thereof which is capable of presenting a target antigen to a T cell, the method comprising contacting an antigen presenting cell or precursor thereof with a CD300f binding protein in the presence of the target antigen for sufficient time to allow uptake of the target antigen by the antigen presenting cell.

In one embodiment, the antigen presenting cell or precursor thereof is contacted with a CD300f binding protein in the presence of the target antigen by contacting the antigen presenting cell or precursor thereof with an immunoconjugate comprising the CD300f binding protein and the target antigen.

In one embodiment, the immunoconjugate is a fusion protein comprising the CD300f binding protein and the target antigen.

In one embodiment, the antigen presenting cell is a dendritic cell (DC). In one embodiment, the dendritic cell is a myeloid dendritic cell (Mo-DC). In one embodiment, the myeloid DC expresses one or more of the markers selected from CD11c, CD1c and CD16. In one embodiment, the myeloid DC expresses CD11c.

In one embodiment, the myeloid DC expresses CD1c. In one embodiment, the myeloid DC expresses CD16. In one embodiment, the myeloid DC expresses CD11c, CD1c and CD16.

In one embodiment, the antigen-loaded antigen presenting cell or precursor thereof is a DC vaccine.

In one embodiment, the precursor of an antigen presenting cell is a monocyte.

In one embodiment, contacting of the antigen presenting cell or precursor thereof with the CD300f binding protein promotes activation of the antigen presenting cell or precursor thereof.

In one embodiment, activation of the antigen presenting cell or precursor thereof increases expression of one or more activation markers.

In one embodiment, the activation marker is one or more proteins selected from CD80, CD83, CD86, and HLA-DR.

In one embodiment, the antigen presenting cell is a dendritic cell and the activation markers are CD80, CD83 and CD86.

The inventors have shown that antigen presenting cells that have been loaded with a target antigen using CD300f binding protein produce an antigen specific T cell response to the target antigen.

Accordingly, in one aspect, there is provided a method of presenting a target antigen to a T cell, the method comprising:
(a) antigen loading an antigen presenting cell or precursor thereof with a target antigen by contacting an antigen presenting cell or precursor thereof with a CD300f binding protein in the presence of the target antigen for sufficient time to allow uptake of the target antigen by the antigen presenting cell or precursor thereof to produce an antigen-loaded antigen presenting cell or precursor thereof;
(b) culturing the antigen-loaded antigen presenting cell or precursor thereof with a population comprising T cells under conditions which promote presentation of the antigen to T cells of the population.

Another aspect provides a method of promoting or increasing a T cell response to a target antigen, the method comprising:
(a) antigen loading an antigen presenting cell or precursor thereof with a target antigen by contacting the antigen presenting cell or precursor thereof with a CD300f binding protein in the presence of the target antigen for sufficient time to allow uptake of the target antigen by the antigen presenting cell or precursor thereof to produce an antigen-loaded antigen presenting cell or precursor thereof;
(b) culturing the antigen-loaded antigen presenting cell or precursor thereof with a population comprising T cells under conditions which promote presentation of the antigen to T cells of the population.

Methods for culturing T cell populations for antigen presentation and T cell stimulation are known in the art.

In one embodiment, the population comprising T cells comprises a lymphocyte population comprising CD3⁺ T cells. In one embodiment, the population comprising T cells comprises a lymphocyte population comprising CD3⁺, CD8⁺ T cells. In one embodiment, the population comprising T cells comprises a lymphocyte population comprising CD3⁺, CD4⁺ and CD8⁺ T cells. In one embodiment, the population comprising T cells comprises cytotoxic T cells. Methods for the preparation of T cell populations are known in the art, and described in, for example, Hsu et al., (2018) A blood dendritic cell vaccine for acute myeloid leukemia expands anti-tumor T cell responses at remission, Oncolmmunology, 7:4, e1419114.

One aspect provides a T cell population comprising T cells that have been activated by culturing with an antigen-loaded antigen presenting cell or precursor thereof described herein.

Antigen-loaded antigen presenting cells or precursors thereof may be administered to a subject to promote or increase a T cell response in a subject.

Accordingly, in one aspect, there is provided a method of presenting a target antigen to a T cell in a subject, the method comprising:
(a) antigen loading an antigen presenting cell or precursor thereof with a target antigen by contacting an antigen presenting cell or precursor thereof with a CD300f binding protein in the presence of the target antigen for sufficient time to allow uptake of the target antigen by the antigen presenting cell or precursor thereof to produce an antigen-loaded antigen presenting cell or precursor thereof;
(b) administering the antigen-loaded antigen presenting cell or precursor thereof to the subject.

Another aspect provides a method of promoting or increasing a T cell response to a target antigen in a subject, the method comprising:
(a) antigen loading an antigen presenting cell or precursor thereof with a target antigen by contacting the antigen presenting cell or precursor thereof with a CD300f binding protein in the presence of the target antigen for sufficient time to allow uptake of the target antigen by the antigen presenting cell or precursor thereof to produce an antigen-loaded antigen presenting cell or precursor thereof;
(b) administering the antigen-loaded antigen presenting cell or precursor thereof to the subject.

### CD300f Binding Protein

The CD300f binding protein may be any protein which specifically binds CD300f and is internalized.

CD300f is a member of the CD300 family of immunoregulatory molecules encoded by a gene complex on chromosome 17q25. It is a transmembrane glycoprotein with a cytoplasmic region and an extracellular domain. The cytoplasmic region contains both inhibitory ITIMs and PI3K phosphorylation sites. There are a number of isoforms of the CD300f molecule, but they all share the same Ig-like extracellular domain.

In one embodiment, the CD300f binding protein is an antibody or antigen binding fragment thereof, which specifically binds to CD300f. In one embodiment, the antibody or antigen binding fragment thereof specifically binds to an extracellular domain of CD300f.

The inventors have previously produced a monoclonal antibody, referred to herein as DCR-2, which specifically binds to the extracellular domain of CD300f. DCR-2 is described in WO 2018/094460, the entirety of which is incorporated herein by reference. A hybridoma producing DCR-2 was deposited at CellBank Australia, 214 Hawkesbury Rd., Westmead, NSW 2145, Australia, under the Budapest Treaty on 27 Sep. 2016 and designated accession number CBA20160029.

In one embodiment, the CD300f binding protein comprises:

(a) a heavy chain variable region which comprises:
(i) an amino acid sequence that is at least 70% identical, typically at least 75%, 80%, 85%, 90%, 95%, 96%, 97%, 98%, 99%, or 100% identical, to the amino acid sequence of the amino acid sequence represented by SEQ ID NO: 1; and/or
(ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and

(b) a light chain variable region which comprises:
(i) an amino acid sequence that is at least 70% identical, typically at least 75%, 80%, 85%, 90%, 95%, 96%, 97%, 98%, 99%, or 100% identical, to the amino acid sequence represented by SEQ ID NO: 5; and/or
(ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8.

In one embodiment, the CD300f binding protein comprises:
(a) a heavy chain variable region which comprises a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and a complementarity determining region 3 (CDR3) that comprises an amino acid sequence that is represented by SEQ ID NO: 4; and
(b) a light chain variable region which comprises a complementarity determining region 1 (CDR1) that comprises an amino acid sequence represented by SEQ ID NO: 6, a complementarity determining region 2 (CDR2) that comprises an amino acid sequence represented by SEQ ID NO: 7, and a complementarity determining region 3 (CDR3) that comprises an amino acid sequence represented by SEQ ID NO: 8.

In one embodiment, the CD300f binding protein comprises: (a) a heavy chain variable region which comprises an amino acid sequence that is at least 70% identical, typically at least 75%, 80%, 85%, 90%, 95%, 96%, 97%, 98%, 99%, or 100% identical, to the amino acid sequence of the amino acid sequence represented by SEQ ID NO: 1, and a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and a complementarity determining region 3 (CDR3) that comprises an amino acid sequence that is represented by SEQ ID NO: 4; and (b) a light chain variable region which comprises an amino acid sequence that is at least 70% identical, typically at least 75%, 80%, 85%, 90%, 95%, 96%, 97%, 98%, 99%, or 100% identical, to the amino acid sequence represented by SEQ ID NO: 5, and comprises a complementarity determining region 1 (CDR1) that comprises an amino acid sequence represented by SEQ ID NO: 6, a complementarity determining region 2 (CDR2) that comprises an amino acid sequence represented by SEQ ID NO: 7, and a complementarity determining region 3 (CDR3) that comprises an amino acid sequence represented by SEQ ID NO: 8.

In one embodiment, the CD300f binding protein comprises a heavy chain variable region which comprises the amino acid sequence represented by SEQ ID NO: 1, and a light chain variable region which comprises the amino acid sequence represented by SEQ ID NO: 5.

TABLE 1
Antibody sequences referred to in the sequence listing

| SEQ ID | |
|---|---|
| NO: | Description |
| 1 | amino acid sequence of heavy chain variable |
| | region of DCR-2 |
| 2 | amino acid sequence of CDR1 of heavy chain |
| | variable region of DCR-2 |
| 3 | amino acid sequence of CDR2 of heavy chain |
| | variable region of DCR-2 |
| 4 | amino acid sequence of CDR3 of heavy chain |
| | variable region of DCR-2 |
| 5 | amino acid sequence of light chain variable |
| | region of DCR-2 |
| 6 | amino acid sequence of CDR1 of light chain |
| | variable region of DCR-2 |
| 7 | amino acid sequence of CDR2 of light chain |
| | variable region of DCR-2 |
| 8 | amino acid sequence of CDR3 of light chain |
| | variable region of DCR-2 |
| 9 | nucleotide sequence of heavy chain variable |
| | region of DCR-2 |
| 10 | nucleotide sequence of light chain variable |
| | region of DCR-2 |
| 11 | codon optimized nucleotide sequence encoding |
| | chimeric antibody heavy chain comprising the |
| | heavy chain variable region of DCR-2 combined |
| | with the constant region of the heavy chain of |
| | human anti-TNP IgG1. |
| 12 | codon optimized nucleotide sequence encoding |
| | chimeric antibody light chain comprising the |
| | light chain variable region of DCR-2 combined |
| | with the constant region of the kappa chain of |
| | human anti-TNP IgG1. |
| 13 | amino acid sequence encoded by SEQ ID NO: 11. |
| 14 | amino acid sequence encoded by SEQ ID NO: 12. |
| 15 | amino acid sequence of humanized heavy chain |
| | variable region of DCR-2 |
| 16 | amino acid sequence of humanised light chain |
| | variable region of DCR-2 |
| 17 | amino acid sequence of DCR2 heavy joining |
| | Region |

The amino acid sequence of the heavy chain variable region (V_{H}) of DCR-2 is represented by the amino acid sequence:

(SEQ ID NO: 1)
M E S G G G L V Q P G G P L K L S C A A S G F G F
S G S W M S W V R Q A P G K G L E W I G Q I N P D
S S T I N Y T P S L K D K F I I R S D N A K N T L
Y L Q I N K V R S E D T A L Y Y C A R R G F F E G
Y S A W F A Y W .

The amino acid sequence of CDR1 of the heavy chain variable region of DCR-2 is represented by the amino acid sequence GFGFSGSW (SEQ ID NO: 2).

The amino acid sequence of CDR2 of the heavy chain variable region of DCR-2 is represented by the amino acid sequence INPDSSTI (SEQ ID NO: 3).

The amino acid sequence of CDR3 of the heavy chain variable region of DCR-2 is represented by the amino acid sequence ARRGFFEGYSAWFAY (SEQ ID NO: 4).

The amino acid sequence of the light chain variable region (V_{L}) of DCR-2 is represented by the amino acid sequence:

(SEQ ID NO: 5)
I L M T Q T P K F L L V S A G D R V T I T C K A
S Q S V S N D V A W Y Q Q K P G Q S P S L L I Y
Y A S N R N T G V P D R F T G S G Y E T D F T F
T I S T V Q A E D L A V Y F C Q Q D Y T S P W T
F G G G .

The amino acid sequence of CDR1 of the light chain variable region of DCR-2 is represented by the amino acid sequence QSVSND (SEQ ID NO: 6).

The amino acid sequence of CDR2 of the light chain variable region of DCR-2 is represented by the amino acid sequence YAS (SEQ ID NO: 7).

The amino acid sequence of CDR3 of the light chain variable region of DCR-2 is represented by the amino acid sequence QQDYTSPWT (SEQ ID NO: 8).

In one embodiment, the CD300f binding protein comprises:

(a) a heavy chain variable region which comprises:

(i) an amino acid sequence that is at least 70% identical, typically at least 75%, 80%, 85%, 90%, 95%, 96%, 97%, 98%, or 99% identical, to the amino acid sequence represented by SEQ ID NO: 1; or

(ii) a complementarity determining region 1 (CDR1) that comprises the amino acid sequence represented by SEQ ID NO: 2, a complementarity determining region 2 (CDR2) that comprises the amino acid sequence represented by SEQ ID NO: 3, and/or a complementarity determining region 3 (CDR3) that comprises the amino acid sequence represented by SEQ ID NO: 4; and

(b) a light chain variable region which comprises:

(i) an amino acid sequence that is at least 70% identical, typically at least 75%, 80%, 85%, 90%, 95%, 96%, 97%, 98%, or 99% identical, to the amino acid sequence represented by SEQ ID NO: 5; or

(ii) a complementarity determining region 1 (CDR1) that comprises the amino acid sequence represented by SEQ ID NO: 6, a complementarity determining region 2 (CDR2) that comprises the amino acid sequence represented by SEQ ID NO: 7, and/or a complementarity determining region 3 (CDR3) that comprises the amino acid sequence represented by SEQ ID NO: 8.

In one embodiment, the CD300f binding protein comprises:

(a) a heavy chain variable region which comprises:

(i) an amino acid sequence that is at least 70% identical, typically at least 75%, 80%, 85%, 90%, 95%, 96%, 97%, 98%, or 99% identical, to the amino acid sequence represented by SEQ ID NO: 1; and/or

(b) a light chain variable region which comprises:

(i) an amino acid sequence that is at least 70% identical, typically at least 75%, 80%, 85%, 90%, 95%, 96%, 97%, 98%, or 99% identical, to the amino acid sequence represented by SEQ ID NO: 5.

In one embodiment, the CD300f binding protein comprises:

(a) a heavy chain variable region which comprises:

(i) a complementarity determining region 1 (CDR1) that comprises the amino acid sequence represented by SEQ ID NO: 2, a complementarity determining region 2 (CDR2) that comprises the amino acid sequence represented by SEQ ID NO: 3, and a complementarity determining region 3 (CDR3) that comprises the amino acid sequence represented by SEQ ID NO: 4; or

(b) a light chain variable region which comprises:

(i) a complementarity determining region 1 (CDR1) that comprises the amino acid sequence represented by SEQ ID NO: 6, a complementarity determining region 2 (CDR2) that comprises the amino acid sequence represented by SEQ ID NO: 7, and a complementarity determining region 3 (CDR3) that comprises the amino acid sequence represented by SEQ ID NO: 8.

In one embodiment, the CD300f binding protein comprises:

(a) a heavy chain variable region which comprises:

(i) a complementarity determining region 1 (CDR1) that comprises the amino acid sequence represented by SEQ ID NO: 2, a complementarity determining region 2 (CDR2) that comprises the amino acid sequence represented by SEQ ID NO: 3, and a complementarity determining region 3 (CDR3) that comprises the amino acid sequence represented by SEQ ID NO: 4; and

(b) a light chain variable region which comprises:

(i) a complementarity determining region 1 (CDR1) that comprises the amino acid sequence represented by SEQ ID NO: 6, a complementarity determining region 2 (CDR2) that is identical to the amino acid sequence represented by SEQ ID NO: 7, and a complementarity determining region 3 (CDR3) that comprises the amino acid sequence represented by SEQ ID NO: 8.

In one embodiment, the CD300f binding protein comprises a heavy chain variable region which comprises an amino acid sequence that is 100% identical to the amino acid sequence represented by SEQ ID NO: 1.

In one embodiment, the CD300f binding protein comprises a light chain variable region which is 100% identical to the amino acid sequence represented by SEQ ID NO: 5.

The inventors have further humanized the heavy and light chain variable regions of DCR-2. The amino acid sequence of the humanized heavy chain variable region (V_{L}) of DCR-2 is represented by the amino acid sequence:

(SEQ ID NO: 15)
M E S G G G L V Q P G G S L R L S C A A S G F G F
S G S W M W S V R Q A P G K G L E W V A N I N P D
S S T I Y Y V D S V K G R F T I S R D N A K N S L
Y L Q M N S L R A E D T A V Y Y C A R R G F F E G
Y S A W F A Y W

The amino acid sequence of the humanized light chain variable region (V_{L}) of DCR-2 is represented by the amino acid sequence:

(SEQ ID NO: 16)
I Q M T Q S P S S L S A S V G D R V T I T C K A
S Q S V S N D L N W Y Q Q K P G K A P K L L I Y
Y A S N L E T G V P S R F S G S G S G T D F T F
T I S S L Q P E D I A T Y Y C Q Q D Y T S P W T
F G G G

In one embodiment, the CD300f binding protein comprises a heavy chain variable region which comprises the amino acid sequence represented by SEQ ID NO: 15, and a light chain variable region which comprises the amino acid sequence represented by SEQ ID NO: 16.

In various embodiments, the CD300f binding protein comprises:
(a) a heavy chain variable region comprising the amino acid sequence represented by SEQ ID NO: 1 or 15;
(b) a heavy chain variable region comprising an amino acid sequence that is at least 70% identical, typically at least 75%, 80%, 85%, 90%, 95%, 96%, 97%, 98%, or 99% identical, to the amino acid sequence represented by SEQ ID NO: 1;
(c) a light chain variable region comprising the amino acid sequence represented by SEQ ID NO: 5 or 16;
(d) a light chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 5;
(e) a heavy chain variable region comprising the amino acid sequence represented by SEQ ID NO: 1, and a light chain variable region comprising the amino acid sequence represented by SEQ ID NO: 5;
(f) a heavy chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 1, and a light chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 5;
(g) a heavy chain CDR1 comprising the amino acid sequence represented by SEQ ID NO: 2;
(h) a heavy chain CDR2 comprising the amino acid sequence represented by SEQ ID NO: 3;
(i) a heavy chain CDR3 comprising the amino acid sequence represented by SEQ ID NO: 4;
(j) a heavy chain CDR1 comprising the amino acid sequence represented by SEQ ID NO: 2 and a heavy chain CDR2 comprising the amino acid sequence represented by SEQ ID NO: 3;
(k) a heavy chain CDR1 comprising the amino acid sequence represented by SEQ ID NO: 2 and a heavy chain CDR3 comprising the amino acid sequence represented by SEQ ID NO: 4;
(l) a heavy chain CDR2 comprising the amino acid sequence represented by SEQ ID NO: 3 and a heavy chain CDR3 comprising the amino acid sequence represented by SEQ ID NO: 4;
(m) a heavy chain CDR1 comprising the amino acid sequence represented by SEQ ID NO: 2, a heavy chain CDR2 comprising the amino acid sequence represented by SEQ ID NO: 3, and a heavy chain CDR3 comprising the amino acid sequence represented by SEQ ID NO: 4;
(n) a light chain CDR1 comprising the amino acid sequence represented by SEQ ID NO: 6;
(o) a light chain CDR2 comprising the amino acid sequence represented by SEQ ID NO: 7;
(p) a light chain CDR3 comprising the amino acid sequence represented by SEQ ID NO: 8;
(q) a light chain CDR1 comprising the amino acid sequence represented by SEQ ID NO: 6, and a light chain CDR2 comprising the amino acid sequence represented by SEQ ID NO: 7;
(r) a light chain CDR1 comprising the amino acid sequence represented by SEQ ID NO: 6, and a light chain CDR3 comprising the amino acid sequence represented by SEQ ID NO: 8;
(s) a light chain CDR2 comprising the amino acid sequence represented by SEQ ID NO: 7, and a light chain CDR3 comprising the amino acid sequence represented by SEQ ID NO: 8;
(t) a light chain CDR1 comprising the amino acid sequence represented by SEQ ID NO: 6, a light chain CDR2 comprising the amino acid sequence represented by SEQ ID NO: 7, and a light chain CDR3 comprising the amino acid sequence represented by SEQ ID NO: 8;
(u) a heavy chain CDR1 comprising the amino acid sequence represented by SEQ ID NO: 2, and a light chain CDR1 comprising the amino acid sequence represented by SEQ ID NO: 6;
(v) a heavy chain CDR2 comprising the amino acid sequence represented by SEQ ID NO: 3, and a light chain CDR2 comprising the amino acid sequence represented by SEQ ID NO: 7;
(w) a heavy chain CDR3 comprising the amino acid sequence represented by SEQ ID NO: 4, and a light chain CDR3 comprising the amino acid sequence represented by SEQ ID NO: 8;
(x) a heavy chain CDR1 comprising the amino acid sequence represented by SEQ ID NO: 2, a heavy chain CDR2 comprising the amino acid sequence represented by SEQ ID NO: 3, a light chain CDR1 comprising the amino acid sequence represented by SEQ ID NO: 6, and a light chain CDR2 comprising the amino acid sequence represented by SEQ ID NO: 7;
(y) a heavy chain CDR1 comprising the amino acid sequence represented by SEQ ID NO: 2, a heavy chain CDR3 comprising the amino acid sequence represented by SEQ ID NO: 4, a light chain CDR1 comprising the amino acid sequence represented by SEQ ID NO: 6, and a light chain CDR3 comprising the amino acid sequence represented by SEQ ID NO: 8;
(z) a heavy chain CDR2 comprising the amino acid sequence represented by SEQ ID NO: 3, a heavy chain CDR3 comprising the amino acid sequence represented by SEQ ID NO: 4, a light chain CDR2 comprising the amino acid sequence represented by SEQ ID NO: 7, and a light chain CDR3 comprising the amino acid sequence represented by SEQ ID NO: 8;
(aa) a heavy chain CDR1 comprising the amino acid sequence represented by SEQ ID NO: 2, a heavy chain CDR2 comprising the amino acid sequence represented by SEQ ID NO: 3, a heavy chain CDR3 comprising the amino acid sequence represented by SEQ ID NO: 4, a light chain CDR1 comprising the amino acid sequence represented by SEQ ID NO: 6, a light chain CDR2 comprising the amino acid sequence represented by SEQ ID NO: 7, and a light chain CDR3 comprising the amino acid sequence represented by SEQ ID NO: 8;
(bb) a heavy chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 1, and comprises a CDR1 comprising the amino acid sequence represented by SEQ ID NO: 2, a CDR2 comprising the amino acid sequence represented by SEQ ID NO: 3, and a CDR3 comprising the amino acid sequence represented by SEQ ID NO: 4;
(cc) a light chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 5, and comprises a CDR1 comprising the amino acid sequence represented by SEQ ID NO: 6, a CDR2 comprising the amino acid sequence represented by SEQ ID NO: 7, and a CDR3 comprising the amino acid sequence represented by SEQ ID NO: 8;
(dd) a heavy chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 1, and comprises a CDR1 comprising the amino acid sequence represented by SEQ ID NO: 2, a CDR2 comprising the amino acid sequence represented by SEQ ID NO: 3, and a CDR3 comprising the amino acid sequence represented by SEQ ID NO: 4, and a light chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 5, and comprises a CDR1 comprising the amino acid sequence represented by SEQ ID NO: 6, a CDR2 comprising the amino acid sequence represented by SEQ ID NO: 7, and a CDR3 comprising the amino acid sequence represented by SEQ ID NO: 8;
(ee) a heavy chain variable region comprising an amino acid sequence that is at least 90% identical, typically at least 95%, 96%, 97%. 98%, or 99% identical, to the amino acid sequence represented by SEQ ID NO: 15, and a light chain variable region comprising an amino acid sequence that is at least 90% identical, typically at least 95%, 96%, 97%. 98%, or 99% identical, to the amino acid sequence represented by SEQ ID NO: 16;
(ff) a heavy chain variable region comprising the amino acid sequence represented by SEQ ID NO: 15, and a light chain variable region comprising the amino acid sequence represented by SEQ ID NO: 16;
(gg) heavy chain variable region comprising the amino acid sequence represented by SEQ ID NO: 15, and a light chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 5, and comprises a CDR1 comprising the amino acid sequence represented by SEQ ID NO: 6, a CDR2 comprising the amino acid sequence represented by SEQ ID NO: 7, and a CDR3 comprising the amino acid sequence represented by SEQ ID NO: 8;
(hh) a heavy chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 1, and comprises a CDR1 comprising the amino acid sequence represented by SEQ ID NO: 2, a CDR2 comprising the amino acid sequence represented by SEQ ID NO: 3, and a CDR3 comprising the amino acid sequence represented by SEQ ID NO: 4, and a light chain variable region comprising the amino acid sequence represented by SEQ ID NO: 16.

In one embodiment, the CD300f binding protein comprises a heavy chain comprising the amino acid sequence represented by SEQ ID NO: 13.

In one embodiment, the CD300f binding protein comprises a light chain comprising the amino acid sequence represented by SEQ ID NO: 14.

In one embodiment, the CD300f binding protein comprises a heavy chain variable region which comprises an amino acid sequence that is at least 90% identical to the amino acid sequence represented by SEQ ID NO: 1 or 15, and a light chain variable region which comprises an amino acid sequence that is at least 90% identical to the amino acid sequence represented by SEQ ID NO: 5 or 16.

In one embodiment, the CD300f binding protein comprises a heavy chain variable region which comprises an amino acid sequence that is at least 95% identical to the amino acid sequence represented by SEQ ID NO: 1 or 15, and a light chain variable region which comprises an amino acid sequence that is at least 95% identical to the amino acid sequence represented by SEQ ID NO: 5 or 16.

In one embodiment, the CD300f binding protein comprises: a heavy chain variable region which comprises a CDR1 comprising the amino acid sequence represented by SEQ ID NO: 2, a CDR2 comprising the amino acid sequence represented by SEQ ID NO: 3, and a CDR3 comprising the amino acid sequence represented by SEQ ID NO: 4; and a light chain variable region which comprises a CDR1 comprising the amino acid sequence represented by SEQ ID NO: 6, a CDR2 comprising the amino acid sequence represented by SEQ ID NO: 7, and a CDR3 comprising the amino acid sequence represented by SEQ ID NO: 8.

In one embodiment, the CD300f binding protein comprises: a heavy chain variable region which comprises an amino acid sequence that is at least 70% identical to the amino acid sequence represented by SEQ ID NO: 1, and comprises a CDR1 comprising the amino acid sequence represented by SEQ ID NO: 2, a CDR2 comprising the amino acid sequence represented by SEQ ID NO: 3, and a CDR3 comprising the amino acid sequence represented by SEQ ID NO: 4; and a light chain variable region which comprises an amino acid sequence that is at least 70% identical to the amino acid sequence represented by SEQ ID NO: 5, and comprises a CDR1 comprising the amino acid sequence represented by SEQ ID NO: 6, a CDR2 comprising the amino acid sequence represented by SEQ ID NO: 7, and a CDR3 comprising the amino acid sequence represented by SEQ ID NO: 8.

In one embodiment, the CD300f binding protein comprises: a heavy chain variable region which comprises an amino acid sequence that is at least 75% identical to the amino acid sequence represented by SEQ ID NO: 1, and comprises a CDR1 comprising the amino acid sequence represented by SEQ ID NO: 2, a CDR2 comprising the amino acid sequence represented by SEQ ID NO: 3, and a CDR3 comprising the amino acid sequence represented by SEQ ID NO: 4; and a light chain variable region which comprises an amino acid sequence that is at least 75% identical to the amino acid sequence represented by SEQ ID NO: 5, and comprises a CDR1 comprising the amino acid sequence represented by SEQ ID NO: 6, a CDR2 comprising the amino acid sequence represented by SEQ ID NO: 7, and a CDR3 comprising the amino acid sequence represented by SEQ ID NO: 8.

The percent identity between two amino acid sequences can be determined using any alignment algorithms known in the art, including for example, the FASTA package of sequence analysis programs (Lipman & Pearson, (1985) Science 227(4693): 1435-1441); BLAST (Altschul et al. J. Mol. Biol. 215(3):403-410.

In one embodiment, the antibody, or antigen binding fragment thereof, binds to human CD300f with an equilibrium dissociation constant (K_{D}) of less than 10⁻⁷M, typically less than 10⁻⁸M, less than 10⁻⁹M, less than 9×10⁻¹⁰M, less than 8×10⁻¹⁰, less than 7×10⁻¹⁰, less than 6×10⁻¹⁰, less than 5×10⁻¹⁰, or less than 4×10⁻¹⁰M.

In one embodiment, the antibody, or antigen binding fragment thereof, binds to human CD300f with an equilibrium dissociation constant (K_{D}) that is in the range of from 1×10⁻¹⁰ to 1×10⁻⁷M, typically in the range of from 1×10⁻¹⁰ to 1×10⁻⁷ M, 1×10⁻¹⁰ to 1×10⁻⁸ M, 1×10⁻¹⁰ to 1×10⁻⁹ M, 1×10⁻¹⁰ to 9×10⁻¹⁰ M, 1×10⁻¹¹ to 8×10⁻¹⁰ M or 1×10⁻¹⁰ to 7×10⁻¹⁰ M, 2×10⁻¹¹ to 6×10⁻¹⁰M, or 3×10⁻¹⁰ to 5×10⁻¹⁰M.

In one embodiment, the antibody, or antigen binding fragment thereof, binds to human CD300f with a K_{A} of about 1×10⁶ M⁻¹s⁻¹ or less. In various embodiments, the antibody or antigen binding fragment thereof binds to human CD300f with a K_{A} in the range of from 1×10⁵ to 5×10⁶ M⁻¹s⁻¹, typically 1×10⁵ to 1×10⁶ M⁻¹s⁻¹.

In one embodiment, the antibody, or antigen binding fragment thereof, binds to human CD300f with an off rate of 5×10⁻² s⁻¹ or less, typically 1×10⁻² s⁻¹ or less, 5×10⁻³ s⁻¹ or less, 1×10⁻³ s⁻¹ or less, or 5×10⁻⁴ s⁻¹ or less, 4×10⁻⁴ s⁻¹ or less, 3×10⁻⁴ s⁻¹ or less, or 2×10⁻⁴ s⁻¹ or less.

In one embodiment, the antibody, or antigen binding fragment thereof, binds to human CD300f with an off rate in the range of from 5×10⁻² s⁻¹ to 1×10⁻⁵ s⁻¹, 1×10⁻² to 5×10⁻⁵ s⁻¹, 5×10⁻³ to 5×10⁻⁴ s⁻¹, 1×10⁻³ to 5×10⁻⁴ s⁻¹, or 1×10⁻³ to 1×10⁻⁴ s⁻¹.

An antibody is an immunoglobulin molecule capable of specifically binding to an antigen. The antibody may be recombinant or modified, including chimeric, humanised, deimmunised, CDR-grafted, synhumanised, bi-specific, or human. A full-length antibody typically comprises two light chains covalently linked to two heavy chains. Each heavy chain of the full-length antibody comprises a heavy chain variable region and a heavy chain constant region. Each light chain of a full-length antibody comprises a light chain variable region and a light chain constant region. Full length antibodies may be any of the following type: IgG, IgM, IgE, IgD, or IgA. In one embodiment, the antibody is IgG.

As used herein, an “antigen binding fragment” of an antibody comprises an antigen binding domain of the antibody, and typically comprises a portion of the antibody that specifically binds the same epitope as the full-length antibody. Typically, the antibody fragment of an antibody comprises portions of the variable region of the heavy and/or the light chain of the antibody. Typically, the antigen binding fragment comprises the CDR1, 2 and/or 3 region of the heavy chain variable region and/or the CDR1, 2 and/or 3 region of the light chain variable region. More typically, the antigen binding fragment comprises the CDR1, 2 and 3 region of the heavy chain variable region and/or the CDR1, 2 and 3 region of the light chain variable region. Still more typically, the antigen binding fragment comprises the CDR1, 2 and 3 region of the heavy chain variable region, and the CDR1, 2 and 3 region of the light chain variable region. In some embodiments, the antigen binding fragment of an antibody comprises the heavy chain variable region and the light chain variable region of an antibody. The portions of the heavy and light chain variable regions may be on separate polypeptide chains, such as Fv fragments, or in a single polypeptide chain in which the light chain and heavy chain variable regions are connected by a peptide linker (“scFv proteins”). Examples of antigen binding fragments of an antibody may include F(ab′)₂, Fab′, Fab, Fv, sFv, scFv, and the like.

As used herein, an antigen binding fragment of an antibody encompasses one or more polypeptides which comprise an antigen binding domain of the antibody, such as an F(ab′)₂, Fab′, Fab, Fv, sFv, or scFv.

An “antigen binding domain” refers to a region of an antibody that is capable of specifically binding to an antigen. Typically, the antigen binding domain comprises CDR1, CDR2 and/or CDR3 from the light chain variable region, and/or CDR1, CDR2 and/or CDR3 from the heavy chain variable region, of an antibody. More typically, the antigen binding domain comprises CDR1, CDR2 and CDR3 from the light chain variable region, and/or CDR1, CDR2 and/or CDR3 from the heavy chain variable region, of an antibody. Still more typically, the antigen binding domain comprises CDR1, CDR2 and CDR3 from the light chain variable region, and CDR1, CDR2 and CDR3 from the heavy chain variable region, of an antibody.

The term “variable region” refers to the portion of the light and/or heavy chain of an antibody that is capable of specifically binding to an antigen. The variable region comprises the complementarity determining regions (CDRs) and the framework regions (FRs). Framework regions are those variable regions other than the complementarity determining regions.

The term “complementarity determining region” refers to one of three amino acid sequences of the variable region of the light chain variable region and/or heavy chain variable region of an antibody that is largely responsible for the ability of the antibody to bind specifically to an antigen. The three complementarity determining regions of the variable region of the light and heavy chain are referred to as CDR1, CDR2 and CDR3.

Methods for determining the CDR regions and the framework regions (FR) of the variable region of the light and heavy chain are known in the art. For example, the amino acid positions assigned to CDRs and FRs may be defined according to Kabat Sequences of Proteins of Immunological Interest, National Institute of Health, Bethesda, Md., 1987 and 1991; Enhanced Clothia Numbering Scheme; Clothia and Lesk J. Mol. Biol. 196:901-917; Clothia et al. Nature 342: 877-883; Honnegher and Plukthun, J. Mol. Biol. 309: 657-670. The antibody, or antigen binding fragment thereof, specifically binds to the extracellular domain of CD300f. As used herein, “an antibody, or antigen binding fragment thereof, that specifically binds to an extracellular domain of CD300f” is an antibody or antigen binding fragment thereof that associates with the extracellular domain of CD300f more frequently, more rapidly, for greater length of time, or with greater affinity, that with other antigens.

The variable domains from antibodies may be cloned using conventional techniques that are known in the art and described in, for example, Sambrook and Russell, Eds, Molecular Cloning: A Laboratory Manual, 3^{rd} Ed, vols. 1-3, Cold Spring Harbor Laboratory Press, 2001. In general, the light chain variable region and heavy chain variable region sequences for antibodies, such as murine antibodies, can be obtained by a variety of molecular cloning procedures, such as RT-PCR, 5′-RACE, and cDNA library screening.

As used herein, a chimeric antibody is an antibody protein that comprises the complementarity determining regions (CDRs), typically the variable regions, of an antibody derived from one species, typically a mouse antibody, while the constant domains of the antibody molecule, and in some embodiments, the framework regions (FR), are derived from another species, such as a human. A humanised antibody is a form of chimeric antibody in which the amino acid sequence of the CDRs is from an antibody from one species; e.g., a mouse antibody, and the amino acid sequence of the constant regions, and typically the framework regions, is from a human antibody.

In one embodiment, the antibody or antigen binding fragment thereof is a chimeric antibody. The chimeric antibody comprises the complementarity-determining regions (CDRs), and typically FR, of DCR-2. The chimeric antibody may comprise the light and heavy chain constant regions of a human antibody. The use of antibody components derived from chimerized monoclonal antibodies reduces potential problems associated with the immunogenicity of murine constant regions. Typically, the antibody is a humanised antibody. Humanization of murine antibodies and antibody fragments is known to those skilled in the art, and described in, for example, U.S. Pat. Nos. 5,225,539; 6,054,297; and 7,566,771. For example, humanized monoclonal antibodies may be produced by transferring murine complementary determining regions from heavy and light variable chains of the mouse immunoglobulin into a human variable domain, and then, substituting human residues in the framework regions of the murine counterparts. The use of human framework region sequences, in addition to human constant region sequences, further reduces the chance of inducing HAMA reactions.

Antibodies can be isolated and purified from serum and hybridoma cultures by a variety of well-established techniques. Such isolation techniques include affinity chromatography with Protein-A Sepharose, size-exclusion chromatography, and ion-exchange chromatography. See, for example, Baines et al., “Purification of Immunoglobulin G (IgG),” in Methods in Molecular Biology, Vol. 10, pages 79-104 (The Humana Press, Inc. 1992).

In some embodiments, an antigen binding fragment of an antibody includes portions of the variable region of the heavy and/or light chain of the antibody. The portions of the heavy chain variable region and/or light chain variable region may be on separate polypeptide chains, such as Fv fragments, or in a single polypeptide chain in which light and heavy variable regions are connected by a peptide linker (e.g. scFv proteins). Examples of antibody fragments include F(ab′)₂, Fab′, Fab, Fv, sFv, scFv, and the like. Typically, the antibody fragment comprises the CDR1, 2 and 3 region of the heavy chain variable region and/or the CDR1, 2 and 3 region of the light chain variable region. Antibody fragments which recognize specific epitopes can be generated by known techniques. F(ab′)₂ fragments, for example, can be produced by pepsin digestion of the antibody molecule. These and other methods are described, for example, by Harlow & Lane (Eds.) Antibodies: A Laboratory Manual, CSHL Press, 1988. Alternatively, Fab′ expression libraries can be constructed to allow rapid and easy identification of Fab′ fragments with the desired specificity.

In some embodiments, an antigen binding fragment of an antibody may be a single chain Fv molecule (scFv). A single chain Fv molecule (scFv) typically comprises a light chain variable region and a heavy chain variable region. The light chain variable region and heavy chain variable region are typically covalently linked by a peptide linker (L) and fold to form an antigen binding site. While the heavy chain variable region and light chain variable region may be directly joined together, those skilled in the art will appreciate that the regions may be separated by a peptide linker consisting of one or more amino acids. Peptide linkers and their use are known in the art. Generally, the peptide linker will have no specific biological activity other than to join the regions or to preserve some minimum distance or other spatial relationship between the heavy chain variable region and light chain variable region. However, the constituent amino acids of the peptide linker may be selected to influence some property of the molecule such as the folding, net charge, or hydrophobicity. Single chain Fv (scFv) antibodies optionally include a peptide linker of no more than 50 amino acids, generally no more than 40 amino acids, preferably no more than 30 amino acids, and more preferably no more than 20 amino acids in length.

Methods of making scFv antibodies are known in the art, and have been described in, for example, U.S. Pat. No. 5,260,203; Lo(Ed), Antibody Engineering: Methods and Protocols (Methods in Molecular Biology, v. 248): p 117-134 and 161-190. In brief, mRNA from B-cells from an immunized animal is isolated and cDNA is prepared. The cDNA is amplified using primers specific for the variable regions of heavy and light chains of immunoglobulins. The PCR products are purified, and the nucleic acid sequences are joined. If a linker peptide is desired, nucleic acid sequences that encode the peptide are inserted between the heavy and light chain nucleic acid sequences. The nucleic acid which encodes the scFv is inserted into a vector and expressed in the appropriate host cell. The scFv that specifically bind to the desired antigen are typically found by panning of a phage display library. Panning can be performed by any of several methods. Panning can conveniently be performed using cells expressing the desired antigen on their surface or using a solid surface coated with the desired antigen. Conveniently, the surface can be a magnetic bead. The unbound phage are washed off the solid surface and the bound phage are eluted.

Methods for preparing other antigen binding fragments of antibodies are known in the art. For example, antigen binding fragments can also be prepared by proteolytic hydrolysis of a full-length antibody or by expression in E. coli or another host of the DNA coding for the fragment. An antibody fragment can be obtained by pepsin or papain digestion of full-length antibodies by conventional methods. For example, an antibody fragment can be produced by enzymatic cleavage of antibodies with pepsin to provide an approximate 100 Kd fragment denoted F(ab′)₂. This fragment can be further cleaved using a thiol reducing agent, and optionally a blocking group for the sulfhydryl groups resulting from cleavage of disulfide linkages, to produce an approximate 50 Kd Fab′ monovalent fragment. Alternatively, an enzymatic cleavage using papain produces two monovalent Fab fragments and an Fc fragment directly.

Other methods of cleaving antibodies, such as separation of heavy chains to form monovalent light-heavy chain fragments, further cleavage of fragments, or other enzymatic, chemical or genetic techniques may also be used, so long as the fragments bind to the epitope that is recognized by the intact antibody.

One aspect provides a CD300f binding protein which comprise a heavy chain variable region comprising the amino acid sequence represented by SEQ ID NO: 15, and a light chain variable region comprising the amino acid sequence represented by SEQ ID NO: 16.

In one embodiment, the CD300f binding protein comprises:

(a) a heavy chain variable region comprising the amino acid sequence represented by SEQ ID NO: 15, and
(b) a light chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 5, and comprises a CDR1 comprising the amino acid sequence represented by SEQ ID NO: 6, a CDR2 comprising the amino acid sequence represented by SEQ ID NO: 7, and a CDR3 comprising the amino acid sequence represented by SEQ ID NO: 8.
In one embodiment, the CD300f binding protein comprises:
(a) a heavy chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 1, and comprises a CDR1 comprising the amino acid sequence represented by SEQ ID NO: 2, a CDR2 comprising the amino acid sequence represented by SEQ ID NO: 3, and a CDR3 comprising the amino acid sequence represented by SEQ ID NO: 4, and
(b) a light chain variable region comprising the amino acid sequence represented by SEQ ID NO: 16.

In one embodiment, the CD300f binding protein comprises a heavy chain variable region which comprises an amino acid sequence that is at least 90% identical to the amino acid sequence represented by SEQ ID NO: 15 and comprises a CDR1 comprising the amino acid sequence represented by SEQ ID NO: 2, a CDR2 comprising the amino acid sequence represented by SEQ ID NO: 3, and a CDR3 comprising the amino acid sequence represented by SEQ ID NO: 4, and a light chain variable region which comprises an amino acid sequence that is at least 90% identical to the amino acid sequence represented by SEQ ID NO: 16 and comprises a CDR1 comprising the amino acid sequence represented by SEQ ID NO: 6, a CDR2 comprising the amino acid sequence represented by SEQ ID NO: 7, and a CDR3 comprising the amino acid sequence represented by SEQ ID NO: 8.

In one embodiment, the CD300f binding protein comprises a heavy chain variable region which comprises an amino acid sequence that is at least 95% identical to the amino acid sequence represented by SEQ ID NO: 15, and a light chain variable region which comprises an amino acid sequence that is at least 95% identical to the amino acid sequence represented by SEQ ID NO: 16.

In one embodiment, the CD300f binding protein comprises: a heavy chain variable region which comprises an amino acid sequence that is at least 95% identical to the amino acid sequence represented by SEQ ID NO: 15, and comprises a CDR1 comprising the amino acid sequence represented by SEQ ID NO: 2, a CDR2 comprising the amino acid sequence represented by SEQ ID NO: 3, and a CDR3 comprising the amino acid sequence represented by SEQ ID NO: 4; and a light chain variable region which comprises an amino acid sequence that is at least 95% identical to the amino acid sequence represented by SEQ ID NO: 16, and comprises a CDR1 comprising the amino acid sequence represented by SEQ ID NO: 6, a CDR2 comprising the amino acid sequence represented by SEQ ID NO: 7, and a CDR3 comprising the amino acid sequence represented by SEQ ID NO: 8.

### Immunoconjugate

The antigen presenting cell or precursor thereof is loaded with a target antigen by contacting the antigen presenting cell or precursor thereof with the CD300f binding protein in the presence of the target antigen.
The CD300f binding protein is in the presence of the target antigen if internalisation of the CD300f binding protein by the antigen presenting cell or precursor thereof results in internalisation of the target antigen.
In various embodiments, the CD300f binding protein may be in the presence of the target antigen when the CD300f binding protein is:
in the presence of a polypeptide comprising the target antigen;
in the presence of a polypeptide consisting essentially of the target antigen; or
in the presence of a polypeptide consisting of the target antigen.
In one embodiment, the CD300f binding protein is not coupled to the target antigen. In such embodiments, the target antigen is a separate molecule from the CD300f binding protein, but during contacting of the antigen presenting cell or precursor thereof with the CD300f binding protein, the molecule comprising the target antigen is sufficiently close to the CD300f binding molecule to allow internalising of the target antigen when the CD300f binding protein is internalised.

In one embodiment, the CD300f binding protein is coupled to the target antigen. In such embodiments, the CD300f binding protein and target antigen form an immunoconjugate. Thus, in one embodiment, the antigen presenting cell or precursor thereof is contacted with a CD300f binding protein in the presence of the target antigen by contacting the antigen presenting cell or precursor thereof with an immunoconjugate comprising the CD300f binding protein and the target antigen. In one embodiment, the immunoconjugate is a fusion protein comprising the CD300f binding protein and the target antigen.

Accordingly, in one embodiment, there is provided an immunoconjugate comprising a CD300f binding protein coupled to a target antigen. In one embodiment, the CD300f binding protein comprises:

(a) a heavy chain variable region which comprises:
(i) an amino acid sequence that is at least 70% identical to the amino acid sequence of the amino acid sequence represented by SEQ ID NO: 1; and/or
(ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and/or

(b) a light chain variable region which comprises:
(i) an amino acid sequence that is at least 70% identical to the amino acid sequence represented by SEQ ID NO: 5; and/or
(ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8.

In one embodiment, there is provided an immunoconjugate comprising a CD300f binding protein comprising a heavy chain variable region which comprises an amino acid sequence that is at least 70%, typically 75%, 80%, 85%, 90%, 95%, 96%, 97%, 98%, or 99% identical to the amino acid sequence represented by SEQ ID NO: 1, and a light chain variable region which comprises an amino acid sequence that is at least 70%, typically 75%, 80%, 85%, 90%, 95%, 96%, 97%, 98%, or 99% identical to the amino acid sequence represented by SEQ ID NO: 5, wherein the CD300f binding protein is coupled to a target antigen.

In one embodiment, there is provided an immunoconjugate comprising a CD300f binding protein comprising a heavy chain variable region which comprises an amino acid sequence represented by SEQ ID NO: 15, and a light chain variable region which comprises an amino acid sequence that is represented by SEQ ID NO: 16, wherein the CD300f binding protein is coupled to a target antigen.

In one embodiment, there is provided an immunoconjugate comprising a CD300f binding protein comprising a heavy chain variable region which comprises an amino acid sequence that is at least 90%, typically at least 95%, 96%, 97%, 98%, or 99% identical to the amino acid sequence represented by SEQ ID NO: 15 and comprises a CDR1 comprising the amino acid sequence represented by SEQ ID NO: 2, a CDR2 comprising the amino acid sequence represented by SEQ ID NO: 3, and a CDR3 comprising the amino acid sequence represented by SEQ ID NO: 4, and a light chain variable region which comprises an amino acid sequence that is at least 90%, typically at least 95%, 96%, 97%, 98%, or 99% identical to the amino acid sequence represented by SEQ ID NO: 16 and comprises a CDR1 comprising the amino acid sequence represented by SEQ ID NO: 6, a CDR2 comprising the amino acid sequence represented by SEQ ID NO: 7, and a CDR3 comprising the amino acid sequence represented by SEQ ID NO: 8, wherein the CD300f binding protein is coupled to a target antigen.

The term “coupled” is used to describe the association of the CD300f binding protein with the target antigen. Any mode of attachment of the CD300f binding protein to the target antigen is suitable, provided the CD300f binding protein is able to bind to CD300f and be internalised with the attached target antigen as intended. The coupling methods according to the present invention include, for example, expression of the immunoconjugate as a fusion peptide comprising the CD300f binding protein or part thereof directly or indirectly fused to the target antigen, direct or indirect attachment of the target antigen to the CD300f binding protein, with or without prior modification of the target antigen and/or the CD300f binding protein, and/or attachment via linkers. Linkers can be categorized functionally into, for example, acid labile, photosensitive, enzyme cleavable linkers, non-cleavable linkers etc.

In one embodiment, the immunoconjugate is a fusion protein comprising the CD300f binding protein and the target antigen. The CD300f binding protein may be fused directly to the target antigen or may be fused indirectly to the target antigen. The CD300f binding protein is fused directly to the target antigen when there is no intervening amino acid sequence between the CD300f binding protein and the target antigen. The CD300f binding protein is fused indirectly to the target antigen when there is an intervening amino acid sequence between the CD300f binding protein and the target antigen. In embodiments where the CD300f binding protein comprises a separate heavy chain and a light chain, the target antigen may be coupled to the heavy chain or the light chain. Typically, the target antigen is coupled to the heavy chain. In such embodiments, the heavy chain variable region or full-length heavy chain can be expressed as a fusion protein comprising the target antigen, and the fusion combined with the CD300f light chain. In other embodiments, the immunoconjugate may be a fusion protein comprising a CD300f scFv and a target antigen.

In one embodiment, the CD300f binding protein is coupled to the target antigen through a linker.

Examples of methods for producing fusion proteins with antibodies are described in, for example, Tsuji, et al. (2011) J Immunol. 186:1218-1227.

Methods for producing fusion proteins are known in the art. Methods for coupling proteins and other molecules to binding proteins such as antibodies are also known in the art.

In some embodiments, the CD300f binding protein is coupled to a polypeptide comprising the target antigen. In such embodiments, the target antigen is a contiguous amino acid sequence that is part of a polypeptide, and the polypeptide comprising the target antigen is processed following internalisation to present the target antigen on the surface of the antigen present cell or precursor thereof. The polypeptide comprising the target antigen may be any length. Typically, the target antigen comprises an amino acid sequence of a length that can be presented on the surface of the antigen presenting cell in the MHC I complex. Typically, the target antigen comprises an amino acid sequence of 8, 9, 10, 11, 12, 13, 14, or 15 contiguous amino acids.

In some embodiments, the CD300f binding protein is coupled to a full-length protein comprising the target antigen. In some embodiments, the CD300f binding protein is coupled to a polypeptide consisting essentially of the target antigen. In such embodiments, the polypeptide comprises the target antigen and may be flanked on either side by 1, 2, 3, 4, or 5 amino acids that are not amino acid sequence that is presented on the surface of the antigen presenting cell or precursor thereof.

In some embodiments the CD300f binding protein is coupled to a polypeptide consisting of the target antigen.

One aspect provides a fusion protein comprising a CD300f binding protein and a target antigen.

### Target Antigen

The target antigen may be any antigen to which a T cell response is required, and typically comprises an amino acid sequence which can be presented by an antigen presenting cell to a T cell in a manner that will promote an antigen specific T cell response. The target antigen comprises an amino acid sequence that is capable of promoting a T cell response. Typically, the target antigen comprises an amino acid sequence that can be loaded into the MHC class I molecules of the antigen presenting cell. In some embodiments, the target antigen is a full-length protein. In some embodiments, the target antigen is a fragment of a full-length protein comprising an amino acid sequence that can be presented by an antigen presenting cell to a T cell in a manner that will promote an antigen specific T cell response.
In one embodiment, the target antigen is a cancer antigen. A cancer antigen as used herein is a compound, such as a peptide or protein, associated with a tumor or cancer cell surface and which comprises an amino acid sequence which is capable of promoting an immune response when presented on the surface of an antigen presenting cell. Cancer antigens include but are not limited to antigens that are recombinantly expressed, an immunogenic portion of, or a whole tumor or cancer. Such antigens can be isolated or prepared recombinantly or by any other means known in the art. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. Examples of cancer antigens are described in Cheever et al. (2009), The Prioritization of Cancer Antigens: A National Cancer Institute Pilot Project for the Acceleration of Translational Research, Clin. Cancer Res. 15(17):5323-5337, and include, for example, WT-1 or an immunogenic fragment thereof, MUC-1 or an immunogenic fragment thereof, LMP2 or an immunogenic fragment thereof, HPV E6 E7 or an immunogenic fragment thereof, EGFRvIII or an immunogenic fragment thereof, HER-2 or an immunogenic fragment thereof, Idiotype or an immunogenic fragment thereof, MAGE A3 or an immunogenic fragment thereof, p53 non-mutant or an immunogenic fragment thereof, NY-ESO-1 or an immunogenic fragment thereof, PSMA, GD2 or an immunogenic fragment thereof, CEA or an immunogenic fragment thereof, MelanA/MART1 or an immunogenic fragment thereof, Ras mutant or an immunogenic fragment thereof, gp100 or an immunogenic fragment thereof, p53 mutant or an immunogenic fragment thereof, Proteinase3 (PR1) or an immunogenic fragment thereof, Bcr-abl or an immunogenic fragment thereof, Tyrosinase or an immunogenic fragment thereof, survivin or an immunogenic fragment thereof, PSA or an immunogenic fragment thereof, hTERT or an immunogenic fragment thereof, Sarcoma translocation breakpoints or an immunogenic fragment thereof, EphA2 or an immunogenic fragment thereof, PAP or an immunogenic fragment thereof, ML-IAP or an immunogenic fragment thereof, AFP or an immunogenic fragment thereof, EpCAM or an immunogenic fragment thereof, ERG or an immunogenic fragment thereof, NA17 or an immunogenic fragment thereof, PAX3 or an immunogenic fragment thereof, ALK or an immunogenic fragment thereof, Androgen receptor or an immunogenic fragment thereof, Cyclin B1 or an immunogenic fragment thereof, Polysialic Acid or an immunogenic fragment thereof, MYCH or an immunogenic fragment thereof, TRP-2 or an immunogenic fragment thereof, RhoC or an immunogenic fragment thereof, GD3 or an immunogenic fragment thereof, Fucosyl GM1 or an immunogenic fragment thereof, mesothelin or an immunogenic fragment thereof, PSCA (prostate stem cell antigen) or an immunogenic fragment thereof, MAGE Al or an immunogenic fragment thereof, sLe(a) or an immunogenic fragment thereof, CYP1B1 or an immunogenic fragment thereof, PLAC1 or an immunogenic fragment thereof, GM3 ganglioside or an immunogenic fragment thereof, BORIS (brother of regulator of imprinted sites) or an immunogenic fragment thereof, Tn or an immunogenic fragment thereof, GloboH or an immunogenic fragment thereof, ETV6-AML or an immunogenic fragment thereof, NY-BR-1 or an immunogenic fragment thereof, RGS5 or an immunogenic fragment thereof, SART3 or an immunogenic fragment thereof, STn or an immunogenic fragment thereof, Carbonic anhydrase or an immunogenic fragment thereof, PAX5 or an immunogenic fragment thereof, LCK or an immunogenic fragment thereof, HMWMAA or an immunogenic fragment thereof, AKAP-4 or an immunogenic fragment thereof, SSX-2 or an immunogenic fragment thereof, sperm fibrous sheath proteins or an immunogenic fragments thereof, XAGW 1 or an immunogenic fragment thereof, B7H3 or an immunogenic fragment thereof, Legumain or an immunogenic fragment thereof, Tie 2 or an immunogenic fragment thereof, Page 4 or an immunogenic fragment thereof, VEGFR2 or an immunogenic fragment thereof, MAD-CT-1 or an immunogenic fragment thereof, FAP or an immunogenic fragment thereof, PDGFR-beta or an immunogenic fragment thereof, or Fos-related antigen or an immunogenic fragment thereof. An immunogenic fragment is a fragment of a protein which can elicit a T cell response. Typically, the immunogenic fragment is at least 8 amino acids in length, more typically at least 9, 10, or at least 11 amino acids in length. Examples of immunogenic fragments are described in, for example, the Cancer Antigenic Peptide Database (https://caped.icp.ucl.ac.be/Peptide/list); Wei et al. 2019, Cancer-Testis Antigen Peptide Vaccine for Cancer Immunotherapy: Progress and Prospects, Transl. Oncol. 12(5):733-738.

Cancer antigens for promoting or increasing a T cell response towards a cancer cell are known in the art and described in, for example, “A listing of human tumor antigens recognized by T cells,” Renkvist N, Castelli C, Robbins P F, Parmiani G. Cancer Immunology Immunotherapy 50: (1) 3-15 Mar. 2001.

In one embodiment, the target antigen is a microbial antigen. A microbial antigen as used herein is an antigen of a microorganism and includes but is not limited to virus, bacteria, parasites, and fungi. Examples of infectious viruses that have been found in humans and from which a microbial antigen may be obtained include but are not limited to: Retroviridae (e.g. human immunodeficiency viruses, such as HIV-1; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis, hepatitis C; Norwalk and related viruses, and astroviruses).

### Antigen Presenting Cells

Antigen presenting cells (APC), such as dendritic cells (DC), can be isolated from any tissue of a subject in which such cells are found. APC (e.g., DC) can be isolated from, for example, bone marrow, blood, peripheral blood mononuclear cells (PBMC), or spleen of a subject.
Antigen presenting cells or precursors thereof may be isolated using any methods know in the art. Any of a number of methods are known in the art for isolation of antigen presenting cells (such as dendritic cells), including repetitive density gradient separation, fluorescence activated cell sorting techniques, positive selection, negative selection, or a combination thereof. Methods for antigen cell isolation are described in, for example, Fromm et al., (2016) CMRF-56+ blood dendritic cells loaded with mRNA induce effective antigen-specific cytotoxic T-lymphocyte responses, OncoImmunology, 5:6, e1168555, DOI: 10.1080/2162402X.2016.1168555; Prue et al., Peptides for Immunotherapy of Metastatic Hormone Refractory Prostate Cancer. J Immunother 2015, 38(2):71-76; Sallusto, F. and A. Lanzavecchia, J Exp Med, 1994. 179(4): p. 1109-18.
For example, antigen presenting cells may be isolated by positively selecting CD14⁺ cells from PBMC obtained from healthy donors. CD14⁺ cells can be obtained by incubating the PBMC with, for example, CD14 microbeads (Miltenyi Biotec, 130-050-201) and separating the microbeads bound to CD14⁺ cells. CD14+ cells can be differentiated into myeloid dendritic cells (MoDC) by incubating CD14⁺ cells in, for example, complete AB media supplemented with GM-CSF and IL-4 for 5 days. MoDC can be loaded with target antigen by incubating with CD300 antibody and target antigen.
As CD300 is restricted in its expression, isolation of dendritic cells is not necessary. In this regard, the CD300f binding protein can be used to target dendritic cells in a mixed population of cells. Accordingly, in some embodiments, dendritic cells may be loaded ex vivo by incubating any tissue, typically PBMC or blood, with CD300f binding protein and the target antigen. In some embodiments, the CD300f binding protein and target antigen, typically in the form of a conjugate, may be administered to the subject without isolation of any tissue or cells from the subject.

### Composition

The CD300f binding proteins, target antigens, CD300f binding immunoconjugates and dendritic cell vaccines described herein may be formulated as compositions, including pharmaceutical compositions.

Accordingly, a further aspect provides a composition, typically a pharmaceutical composition, comprising:

(a) a CD300f binding protein and a target antigen as described herein; or

(b) an immunoconjugate comprising a CD300f binding protein coupled to a target antigen as described herein; or

(c) an antigen-loaded antigen presenting cell or precursor thereof (e.g., a DC vaccine) as described herein,

and typically, a pharmaceutically acceptable carrier.

A “pharmaceutically acceptable carrier” means that it is compatible with the other ingredients of the composition and is not deleterious to a subject. The compositions may contain other therapeutic agents as described below, and may be formulated, for example, by employing conventional liquid vehicles or diluents, as well as pharmaceutical additives of a type appropriate to the mode of desired administration (for example, excipients, binders, preservatives, stabilizers, flavours, etc.) according to techniques such as those well known in the art of pharmaceutical formulation (See, for example, Remington: The Science and Practice of Pharmacy, 21st Ed., 2005, Lippincott Williams & Wilkins).

The pharmaceutical compositions are typically in the form of a sterile injectable aqueous suspension. This suspension may be formulated according to the known art and contain the active materials in admixture with excipients suitable for the manufacture of aqueous suspensions. Such excipients may include suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectable formulations.

### DC Vaccine and Treatment

The inventors envisage that the methods described herein can be used to prepare a dendritic cell vaccine which can be used to raise an immune response to a target antigen or antigens.

Accordingly, in one embodiment, there is provided a dendritic cell vaccine for promoting a T cell response to a target antigen, the vaccine comprising an antigen-loaded dendritic cell or precursor thereof, wherein the antigen-loaded dendritic cell or precursor thereof is produced by contacting a dendritic cell or precursor thereof with a CD300f binding protein in the presence of a target antigen or antigens.
In one embodiment, the dendritic cell or precursor thereof is contacted with a CD300f binding protein in the presence of a target antigen by contacting the dendritic cell or precursor thereof with a CD300f binding protein conjugate comprising a CD300f binding protein coupled to a target antigen.
Also provided is a method of promoting a T cell response to a target antigen in a subject, the method comprising administering to the subject an effective amount of the dendritic cell vaccine described herein.
Another aspect provides a method of treating a disease or condition requiring a T cell response to a target antigen, the method comprising administering an effective amount of the dendritic cell vaccine described herein.
It is also envisaged that administration of the immunoconjugate described herein can be used to promote an antigen specific immune response to target antigen. In this regard, the target antigen can be delivered or loaded into the DC in vivo. This can be achieved by administering an immunoconjugate in which the CD300f binding protein is coupled to the target antigen. The feasibility of this approach has been demonstrated in Phase 1 clinical trials targeting DEC-205. In this regard, administration of ant-DEC-205 antibodies coupled to a cancer antigen have been shown to produce an anti-tumour immune response. However, DEC-205 has a broad expression profile, and targeting DEC-205 causes tolerance unless co-administered with adjuvant.

One aspect provides a method of promoting or increasing a T cell response to a target antigen in a subject, the method comprising administering an effective amount of an immunoconjugate comprising a CD300f binding protein and the target antigen.

Another aspect provides a method of treating a disease or condition requiring a T cell response to a target antigen in a subject, the method comprising administering an effective amount of a CD300f binding protein conjugate comprising a CD300f binding protein and the target antigen.

As used herein, a disease or condition requiring a T cell response to a target antigen is a disease or condition for which a T cell response to the target antigen may be beneficial.

The methods described herein can be used to treat any disease or condition for which a T cell response to a target antigen may be beneficial.

In one embodiment, the disease or condition is cancer. In such embodiments, the target antigen will be a cancer antigen. Examples of cancer antigens are described in Cheever et al. (2009), The Prioritization of Cancer Antigens: A National Cancer Institute Pilot Project for the Acceleration of Translational Research, Clin. Cancer Res. 15(17):5323-5337, and include, for example, WT-1, MUC-1, LMP2, HPV E6 E7, EGFRvIII, HER-2, Idiotype, MAGE A3, p53 non-mutant, NY-ESO-1, PSMA, GD2, CEA, MelanA/MART1, Ras mutant, gp100, p53 mutant, Proteinase3(PR1), Bcr-abl, Tyrosinase, survivin, PSA, hTERT, Sarcoma translocation breakpoints, EphA2, PAP, ML-IAP, AFP, EpCAM, ERG, NA17, PAX3, ALK, Androgen receptor, Cyclin B1, Polysialic Acid, MYCH, TRP-2, RhoC, GD3, Fucosyl GM1, mesothelin, PSCA (prostate stem cell antigen), MAGE Al, sLe(a), CYP1B1, PLAC1, GM3 ganglioside, BORIS (brother of regulator of imprinted sites), Tn, GloboH, ETV6-AML, NY-BR-1, RGS5, SART3, STn, Carbonic anhydrase, PAX5, LCK, HMWMAA, AKAP-4, SSX-2, sperm fibrous sheath proteins, XAGW 1, B7H3, Legumain, Tie 2, Page 4, VEGFR2, MAD-CT-1, FAP, PDGFR-beta, Fos-related antigen, or an immunogenic fragment thereof. An immunogenic fragment is a fragment of a protein which can elicit a T cell response. Typically, the immunogenic fragment is at least 8 amino acids in length, more typically at least 9, 10, or at least 11 amino acids in length. Examples of immunogenic fragments are described in, for example, the Cancer Antigenic Peptide Database (https://caped.icp.ucl.ac.be/Peptide/list); Wei et al. 2019, Cancer-Testis Antigen Peptide Vaccine for Cancer Immunotherapy: Progress and Prospects, Transl. Oncol. 12(5):733-738.

The pharmaceutical compositions described herein may be administered by any suitable means, typically, parenterally, such as by subcutaneous, intravenous, intramuscular, intra(trans)dermal, or intracisternal injection or infusion techniques (e.g., as sterile injectable aqueous solutions or suspensions); in dosage unit formulations containing non-toxic, pharmaceutically acceptable vehicles or diluents.

The pharmaceutical compositions for the administration may conveniently be presented in unit dosage form and may be prepared by any of the methods well known in the art of pharmacy. In general, the pharmaceutical compositions are prepared by uniformly and intimately bringing the compound into association with a liquid carrier. In the pharmaceutical composition the active compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

Generally, the term “treating” means affecting a subject, tissue or cell to obtain a desired pharmacological and/or physiological effect and include: (a) preventing the disease from occurring in a subject that may be predisposed to the disease, but has not yet been diagnosed as having it; (b) inhibiting the disease, i.e., arresting its development; or (c) relieving or ameliorating the effects of the disease, i.e., cause regression of the effects of the disease. In one embodiment, treatment achieves the result of reducing the number of cancer cells in the recipient subject.

The term “subject” refers to any animal having a disease which requires treatment by the present method. In addition to primates, such as humans, a variety of other mammals can be treated using the methods of the present invention. For instance, mammals including, but not limited to, cows, sheep, goats, horses, dogs, cats, guinea pigs, rats or other bovine, ovine, equine, canine, feline, rodent or murine species can be treated. Dogs in particular are known to experience multiple myeloma.

The term “effective amount” refers to the amount of the antibody, antigen binding fragment or immunoconjugate that will elicit the biological or medical response of a tissue, system, animal or human that is being sought by the researcher, veterinarian, medical doctor or other clinician.

In the treatment or prevention of cancer, an appropriate dosage level of CD300f binding protein conjugate will generally be about 0.01 to 50 mg per kg patient body weight per day which can be administered in single or multiple doses. Preferably, the dosage level will be about 0.1 to about 25 mg/kg per day; more preferably about 0.5 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.05 to 0.5, 0.5 to 5 or 5 to 15 mg/kg per day.

An appropriate dosage level of antigen-loaded dendritic cells per kg patient body weight per day which can be administered in single or multiple doses.

It will be understood that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Also disclosed herein is a kit comprising the CD300f binding protein and target antigen described herein, CD300f binding protein conjugate described herein, or the dendritic cell vaccine described herein. In various embodiments, the kit comprises:

(a) a CD300f binding protein and a target antigen as described herein; or

(b) a CD300f binding protein conjugate comprising a CD300f binding protein coupled to a target antigen as described herein; or

(c) an antigen-loaded antigen presenting cell (e.g., a DC vaccine) as described herein,

It is to be understood that the terminology used herein is for the purpose of describing particular embodiments only and is not intended to limit the scope of the present invention which will be limited only by the appended claims. As used herein and in the appended claims, the singular forms “a”, “an”, and “the” include plural reference unless the context clearly indicates otherwise. Thus, for example, a reference to “an antibody” includes a plurality of such antibodies. Unless defined otherwise, all technical and scientific terms used herein have the same meaning as commonly understood by one of ordinary skill in the art to which this invention belongs.

Although any materials and methods similar or equivalent to those described herein can be used to practice or test the present invention, preferred materials and methods are described herein.

All publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

All publications mentioned in this specification are herein incorporated by reference. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

In order to exemplify the nature of the present invention such that it may be more clearly understood, the following non-limiting examples are provided.

### Examples

The inventors have demonstrated that blood DC are present in localised and advanced disease and assessed the functional contribution of CD300f.

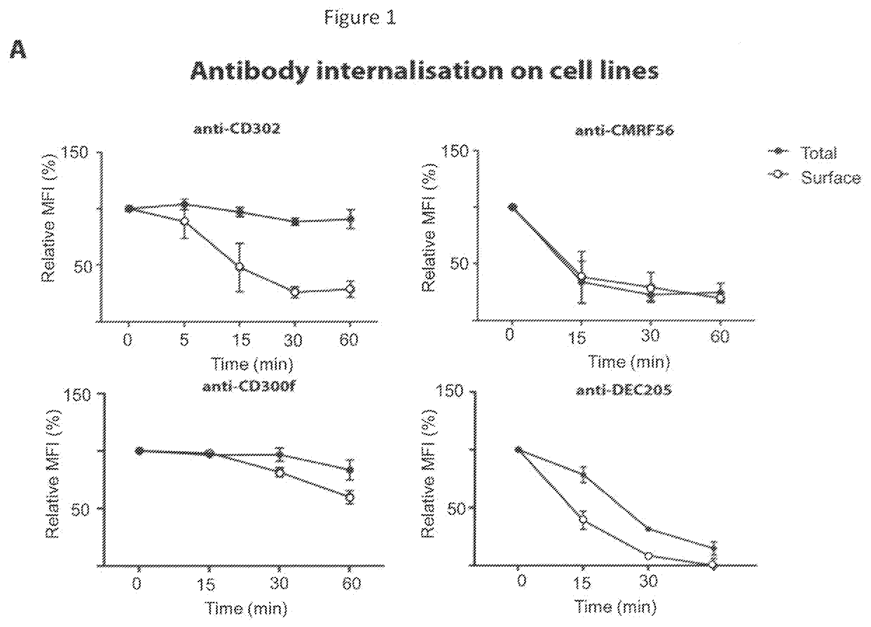

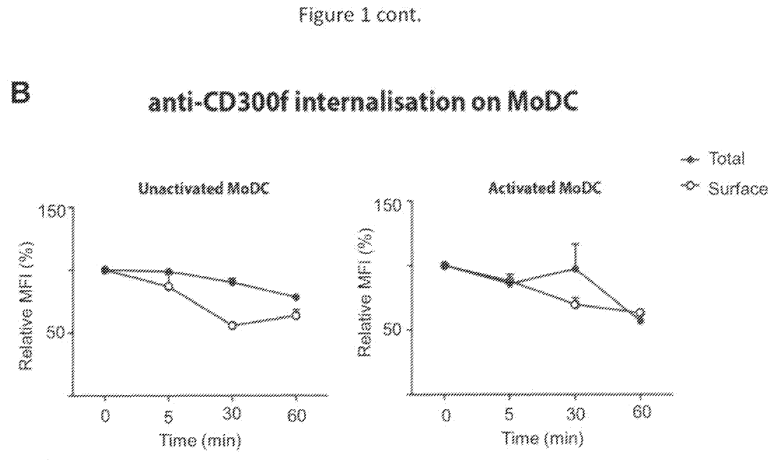

By FACS analysis the inventors assessed three markers that are expressed on DC and other antigen presenting cells for their ability to internalize on cell lines compared to DEC 205. The three chosen markers and their antibodies are DCR-2 (a mouse anti-human CD300f monoclonal antibody), MMRI-20 (a mouse anti-human CD302 monoclonal antibody) and CMRF-56 (a mouse monoclonal antibody that selects for activated DC and monocytes). Internalisation of surface antibody at 37° C. was assessed by comparing total antibody over time (Total, closed symbol) measured using PE labelled MMRI20 and DCR-2 on HL60, a monocytic cell line, and FITC labelled anti-CMRF56 and MMRI-7 on KMH2. This was compared with remaining surface Ig detected with goat anti-mouse IgG AF647 (Invitrogen, A21237). The results are shown in FIG. 1A. As can be seen from FIG. 1A, the inventors found that DCR-2 and MMRI20 internalize, but not CMRF-56. The inventors also looked at the effect of activation on internalisation kinetics. In this regard, the effect of activation of the monocyte derived DC (Mo-DC) on CD300f rate of internalisation was assessed on monocyte derived DC (Mo-DC)+/−LPS 100 ng/ml overnight as an activation agent. These results are shown in FIG. 1B. As can be seen from FIG. 1B, internalisation looks to be reduced with activation of Mo-DC.

Using confocal imaging, the internalization of a polyclonal anti-CD300f antibody, gLMIR3, was compared in fixed cells (and therefore no internalization) and live HL-60 and CD14⁺ monocytes following 30 minutes incubation at 37° C. The results indicated that the CD300f polyclonal antibody gLMIR3 is internalized in live HL-60 and CD14⁺ cells, but not in fixed cells.

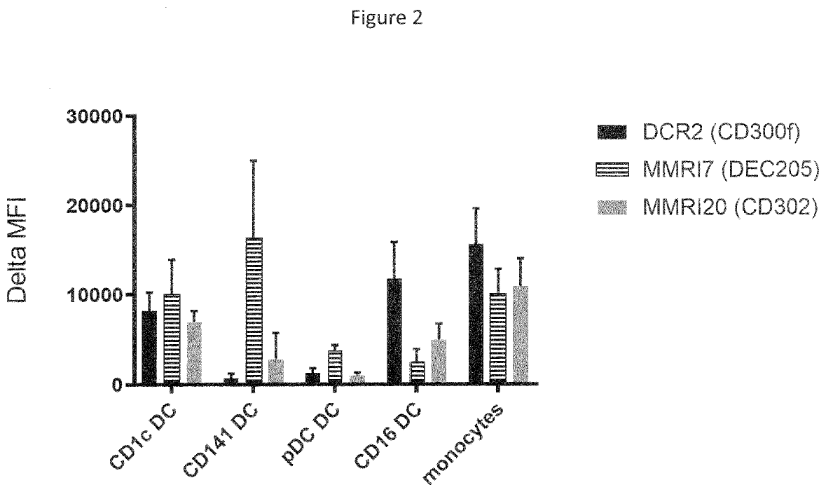

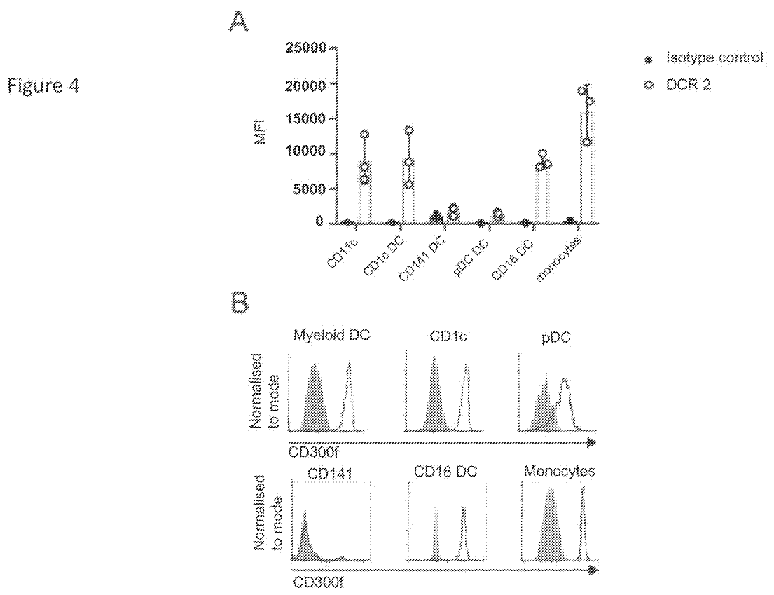

The inventors then compared the expression of CD300f, CD302 and DEC 205 (FIG. 2). In this regard, PBMC were isolated from the peripheral blood of 3 healthy donors. Dendritic cell subsets where determined by FACs analysis using an antibody panel which included antibodies against leucocyte lineages, HLA-DR and CD1c, CD141, pDC, and CD16 as described in Fromm, Kupresanin, Brooks, Dunbar, Haniffa, Hart and Clark, Clinical & Translational Immunology (2016) 5, e68. Expression of CD300f, CD302 and DEC205 was determined by staining on ice for 20 min with biotinylated antibodies against each antigen and then strep PE 1:100 (BD 54061) for 20 min on ice. The results of staining are shown in FIGS. 2 and 4. The results show that both CD300f, DEC205 and CD302 are expressed on CD1c to a similar degree, the main DC subset in peripheral blood that can direct a broad range of T cell responses. CD300f and CD302 are not expressed on CD141 DC. CD300f was highly expressed on CD11c, CD1c and CD16 DC and monocytes (FIGS. 2 and 4A and B).

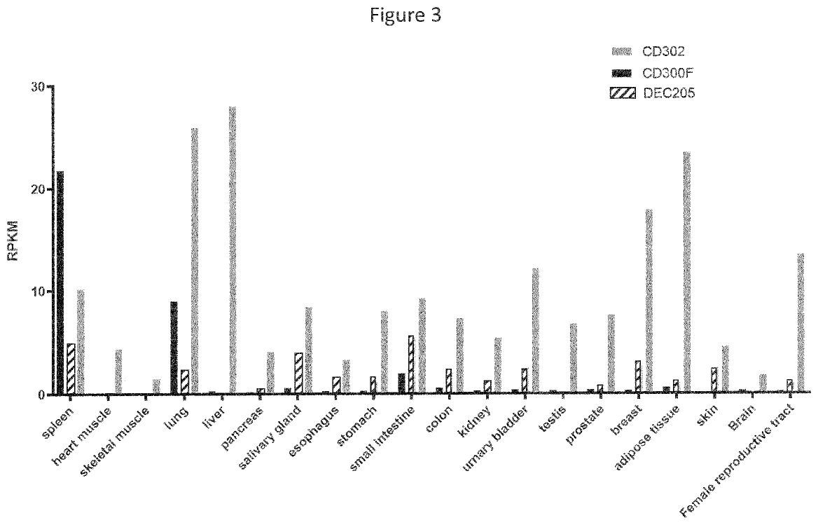

In a target for immunotherapy, high expression in lymphoid tissues such as the spleen and low expression elsewhere is desirable. mRNA expression of CD300f, CD302 and DEC-205 was therefore assessed across normal tissue by RNA sequencing. The results are shown in FIG. 3. As can be seen from FIG. 3, CD300f has a restricted expression profile while CD302 does not (FIG. 3).

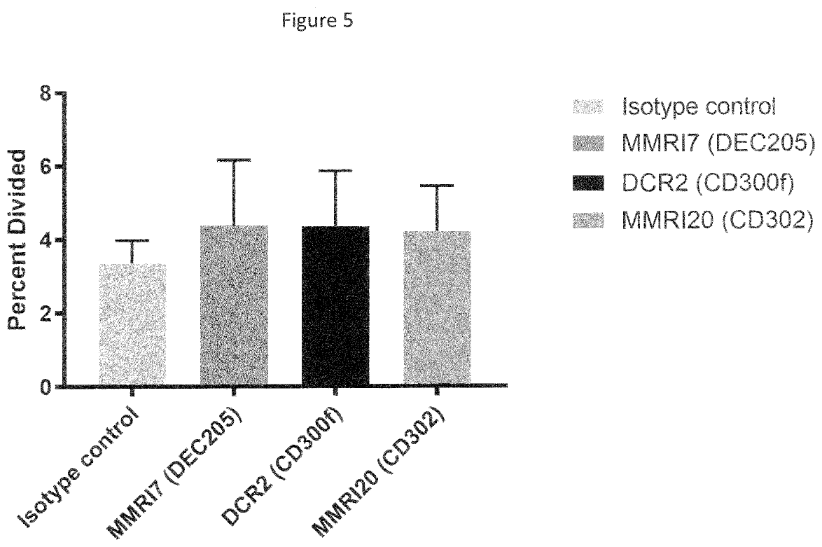

The inventors then assessed whether antibodies against DEC-205 (MMRI7), CD300f (DCR-2) and CD302 (MMRI-20) produce an antigen specific response. In this regard, CD14⁺ cells were positively selected from PBMC from 3 healthy donors by staining PBMC with CD14 microbead (Miltenyi Biotec, 130-050-201) for 15 min at 4° C. and magnetic separation using an autoMACS separator. CD14⁺ cells where differentiated into MoDC by incubating CD14⁺ cells at 3.33×10⁶/mL in complete AB media supplemented with GM-CSF 800 U/mL and IL-4 1000 u/mL for 5 days. CD3 positive cells were isolated from PBMC using EasySep Human T cell Isolation Kit (Stemcell, 17951) and frozen on day 1. MoDC were loaded with CMV antigen on day 5 by staining with primary biotinylated antibody for 20 min on ice and then stained with HCMV-pp65 delivery reagent (Miltenyi Biotec 130-095-406) for 10 mins, washed and then incubated overnight with LPS 100 ng/ml. Autologous T cells were then thawed, rested for 2 hours and stained with CSFE before being added to the Mo-DC at a ratio of 1:10 for 5 days. At day 5 cells were harvested and ran on FACs. The results are shown in FIG. 5. The graph in FIG. 5 shows percent divided (percentage of CD3 CSFE low T cells). As can be seen from FIG. 5, it was found that DCR-2 and MMRI-20 produced a similar response to that of MMRI-7 (FIG. 5).

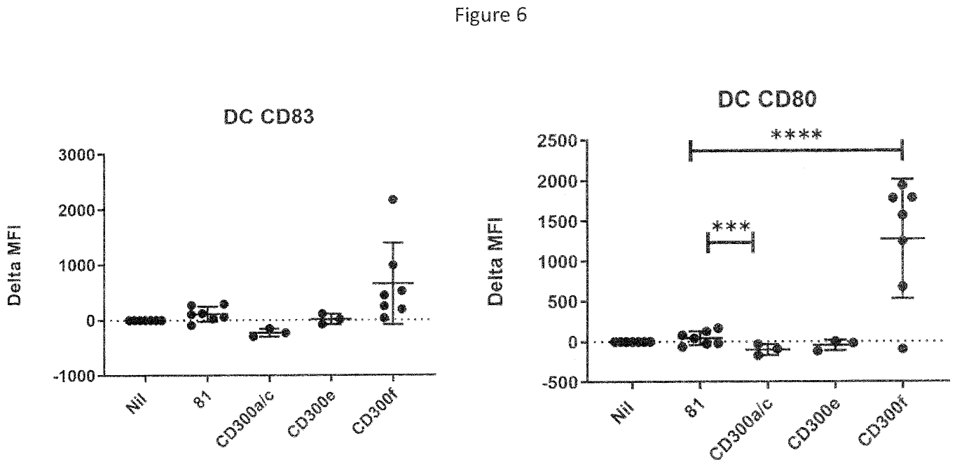

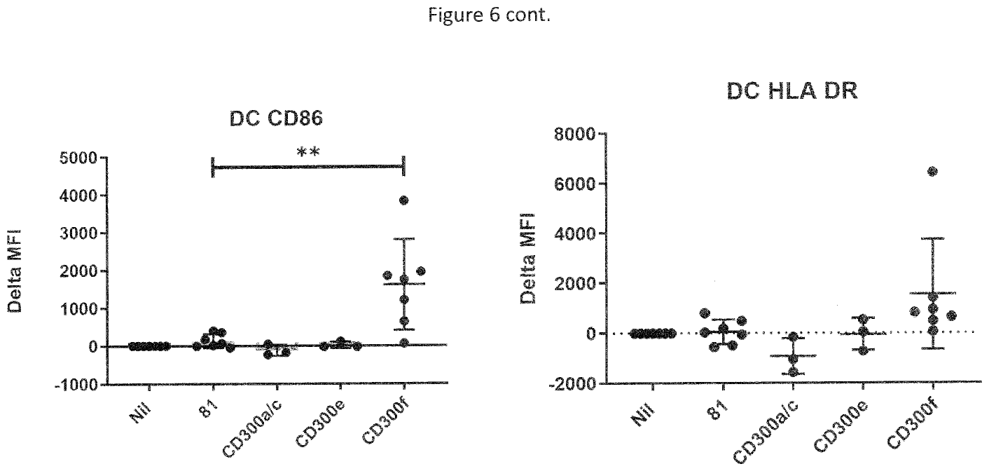

CD300f looked like a good candidate for antigen loading of DC. It was previously shown that the binding of DCR-2 to CD300f enhances the binding of UPD2 (WO 2018/094460) suggesting it is an active antibody that leads to a structural change in CD300f, opening the epitope up for UPD2 to bind. Thus, the inventors assessed whether DCR2 lead to phenotypic changes in myeloid DC. Initially the inventors did this with crosslinking experiments, looking at the effect of crosslinking DCR-2 on myeloid DC and monocytes. 96 well flat bottom tissue culture plates (Fal 353072) were coated with 10 ug/ml of DCR-2 (anti-CD300f), UPH2 (anti-CD300e, Biolegend, 339702), control CMRF81 antibody and CMRF35 (anti CD300a/c) supernatant overnight at 4° C. then washed with PBS. Myeloid DC isolated using EasySep Human Myeloid DC Enrichment Kit (Stem Cell Technologies, 19061) and 1×10⁵ myeloid DC were added in 200 μl of RPMI 10% AB complete media incubated at 37° C. and 5% CO₂ for 18 hours. Supernatant was collected for cytokine analysis and cells harvested for flow cytometry analysis using CD80 Pe-Cy7 (L307.4, BD561135), CD83 FITC (Hb15a, IM2410U), CD86 BV650 (IT2.2, Biolegend) and HLADR APC-H7 (1243, Biolegend). The results are shown in FIG. 6, which shows that crosslinking of DCR-2 leads to upregulation of activation markers on myeloid DC.

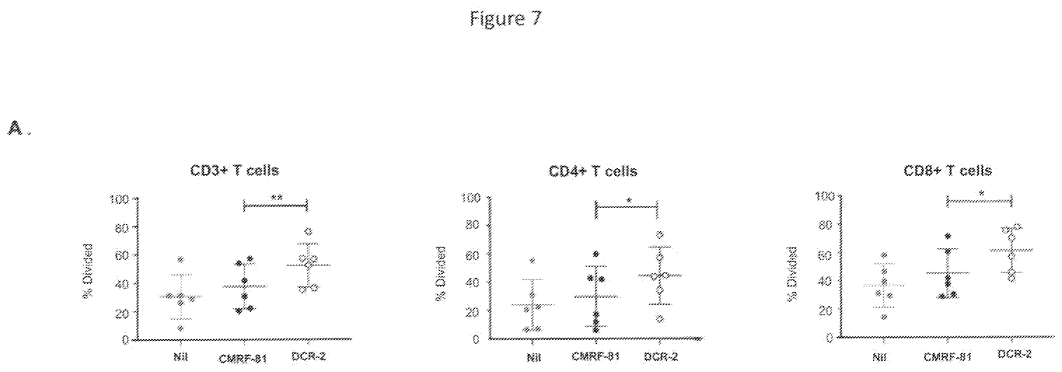

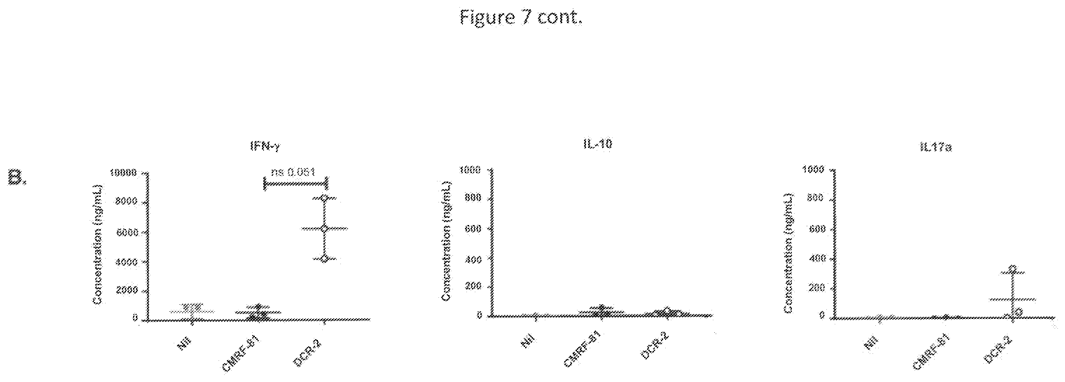

Crosslinking DC with DCR-2 leads to upregulation of activation markers. The inventors also assessed whether DCR-2 changed how myeloid DC responded to LPS and did not find any difference. If DCR-2 leads to increase in activation markers, the inventors asked the questions are these DC more stimulatory. Using an allogenic MLR the inventors show a trend towards these DC being more stimulatory. Myeloid DC from 3 healthy donors were crosslinked overnight as described above in relation to FIG. 6. The myeloid DCs were harvested and added to thawed CSFE labelled CD3 T cells from a single donor and incubated in RPMI 10% AB media for 6 days. At day 6 cells were harvest and analysed by flow cytometry. Stained with CD3 AF 700 (SP34-2, BD 55917), CD4 PerCP Cy5.5 (RPA-T4, BD 560650), CD8 BV421 (RPA-T8, BD 562428). Percent divided determine by frequency of CSFE low cells in each population. The results are shown in FIG. 7A.

The inventors also measured the concentration of cytokines IFN-γ, IL-10 and IL-17a in supernatants of T cells incubated with myeloid DC crosslinked with DCR-2 or CMRF-81. Supernatant was collected at day 6 and cytokine concentration analysed (n=3) using LEGENDplex Human Inflammation Panel Kit (Cat. No. 740409). The results are shown in FIG. 7B. FIG. 7B shows that crosslinking of myeloid DC with DCR-2 promoted production of IFN-γ, and to a lesser extent IL-17a.

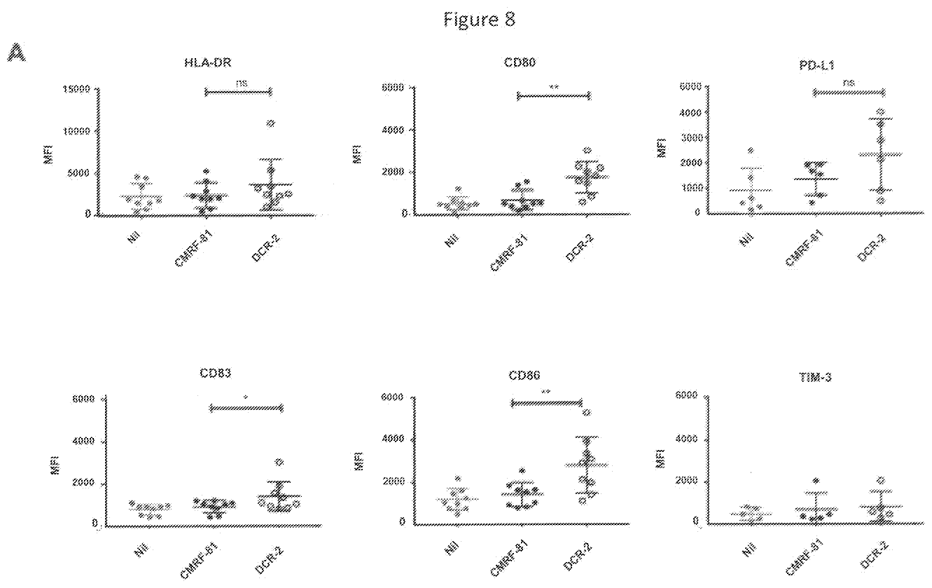

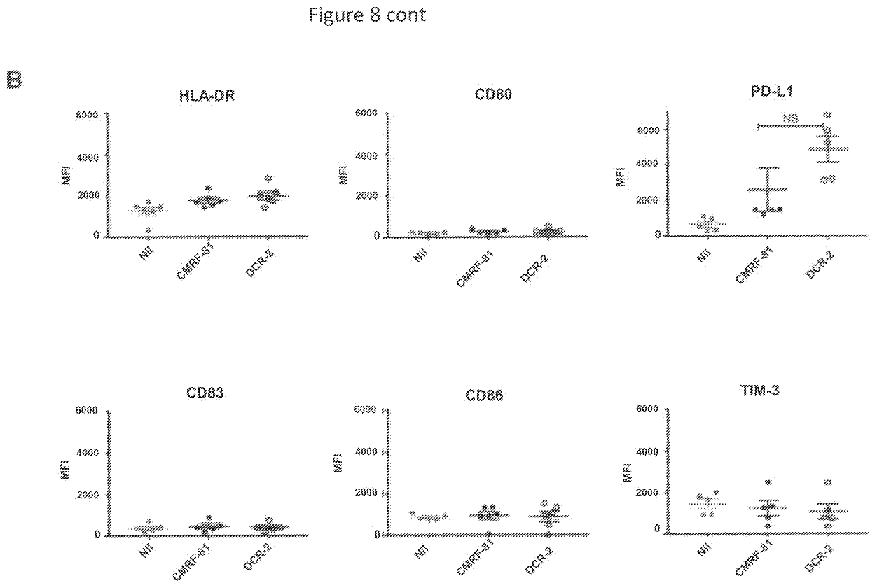

The inventors then asked the question is crosslinking required for DCR-2 to activate DC. The inventors looked at the effect of crosslinking with DCR2 and CMRFI-81 on expression of activation markers on myeloid DC and monocytes. HLA-DR, CD80, PD-L1, CD83, CD86 or TIM-3 on myeloid DC was assessed using flow cytometry following isolation of myeloid DC. Myeloid DC were isolated using EasySep Human Myeloid DC Enrichment Kit (Stem Cell Technologies, 19061) and 1×10⁵ myeloid DC were added in 200 μl of RPMI 10% AB complete media incubated at 37° C. and CO₂ for 18 hours with 10 pg/ml of DCR-2 or control CMRF-81 antibody. Supernatant was collected for cytokine analysis and cells harvested for flow cytometry analysis using CD80 Pe-Cy7 (L307.4, BD561135), CD83 FITC (Hb15a, IM2410U), CD86 BV650 (IT2.2, Biolegend) and HLA-DR APC-H7 (1243, Biolegend). The results are shown in FIG. 8A. HLA-DR, CD80, PD-L1, CD83, CD86 or TIM-3 expression on monocytes was assessed using flow cytometry following isolation of monocytes using magnetic separation and CD14 Microbeads (130-050-201), and crosslinking for 18 hours with CMRF-81 or DCR-2. The results are shown in FIG. 8B. Crosslinking with DCR-2 resulted in statistically significant increases in expression of CD80, CD83, and CD86 in myeloid DC compared to isotype control.

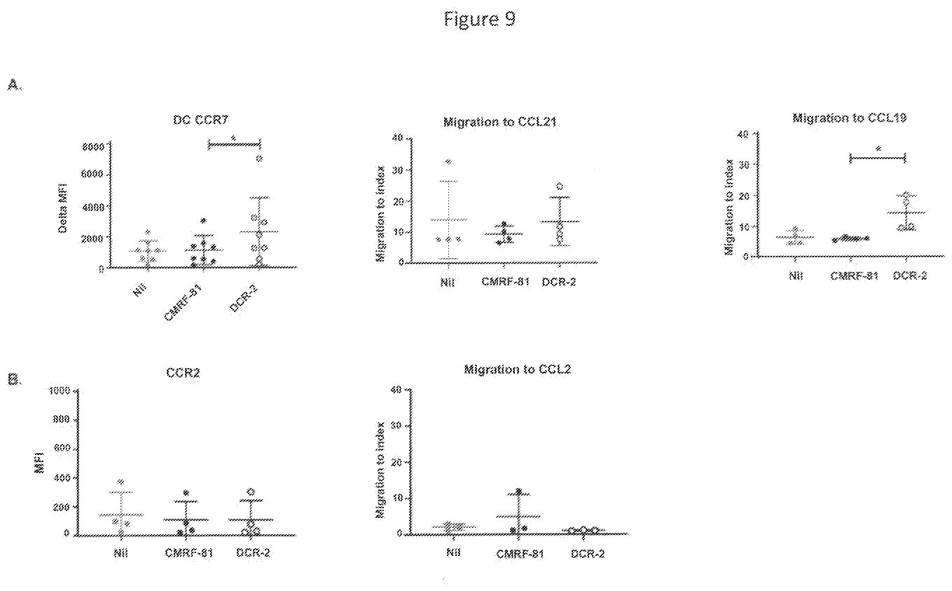

The inventors next tested whether crosslinking with DCR-2 affects migration. First, the inventors assessed expression of the chemokine receptor CCR7. Crosslinked myeloid DC were harvested and stained with CCR7 PE (R&D, FAB197P) for 15 min at 37° C. Migration of crosslinked DC or monocytes was assessed by determining number of DC or monocytes that migrated across transwells (Corning, 3421) towards CCL19 at a concentration of 0.1 ug/ml in 4 hours or CCL21 at 100 ng/ml in 2 hours, or towards CCL2 in 2 hours, all in RPMI (1% BSA, PSG) compared to no chemokine. DC were harvested from the bottom of the transwell stained with Lin2 FITC (CD3/14/19/20/56, BD 643397) and HLA-DR APC-H7 (I243, Biolegend) and resuspended in 200 ul FACS buffer with Count bright Absolute Count Beads 2500/ml. Number of cells migrated was calculated as number of (lineage-DR+ cells/counted beads)×5000 beads. Migration index=# cells migrated chemokine/# cells migrated no chemokine. The results of CCR7 expression in myeloid DC and migration of myeloid DC toward CCL21 and CCL19 following crosslinking with DCR-2 or CMRF-81 are shown in FIG. 9A. As shown in FIG. 9A, CCR7 expression was upregulated on CD300f crosslinked myeloid DC. Migration towards CCL19 was significantly increased by crosslinking with DCR-2 but not towards CCL21. The results of CCR2 expression in monocytes and migration of monocytes towards CCL2 following crosslinking with DCR-2 or CMRF-81 is shown in FIG. 9B.

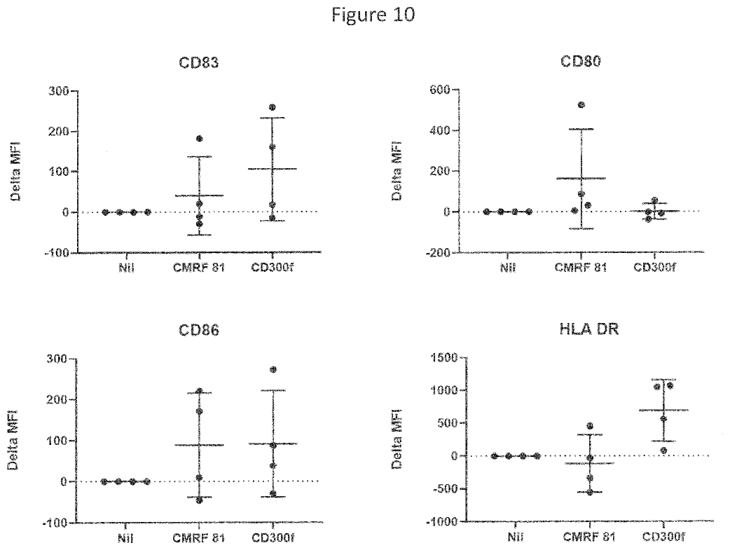

The inventors also assessed the effect of crosslinking on expression of activation markers on monocytes. In this regard, 96 well flat bottom tissue culture plates (Fal 353072) were coated with 10 μg/ml of DCR 2 (anti-CD300f), control CMRF-81 antibody and PBS control at 4° C. then washed with PBS. CD14+ cells were positively selected from N=4 healthy donors using CD14+ microbeads (Miltenyi Biotec 130-050-201) and magnetic separation using AutoMacs. 1×10⁵ CD14⁺ cells were added in 200 μl of RPMI 10% AB complete media incubated at 37° C. and 5% CO2 for 18 hours. Supernatant was collected for cytokine analysis and cells harvested for flow cytometry analysis using CD80 Pe-Cy7 (L307.4, BD561135), CD83 FITC (Hb15a, IM2410U), CD86 BV650 (IT2.2, Biolegend) and HLADR APC-H7 (1243, Biolegend). Results are shown in FIG. 10. In contrast to the DC there is a trend for upregulation of HLA-DR but no other activation markers on monocytes.

Humanised DCR-2

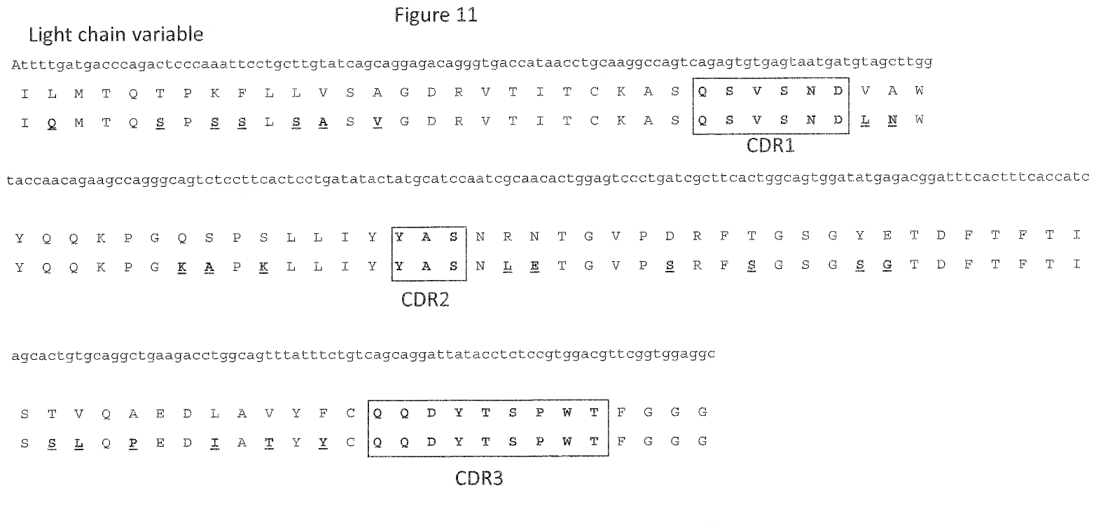

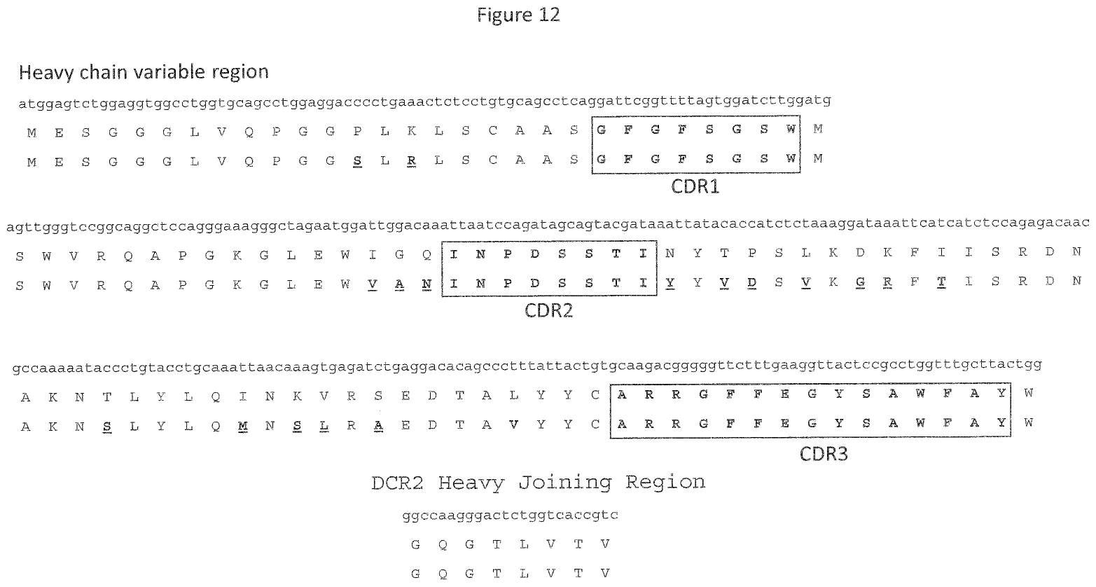

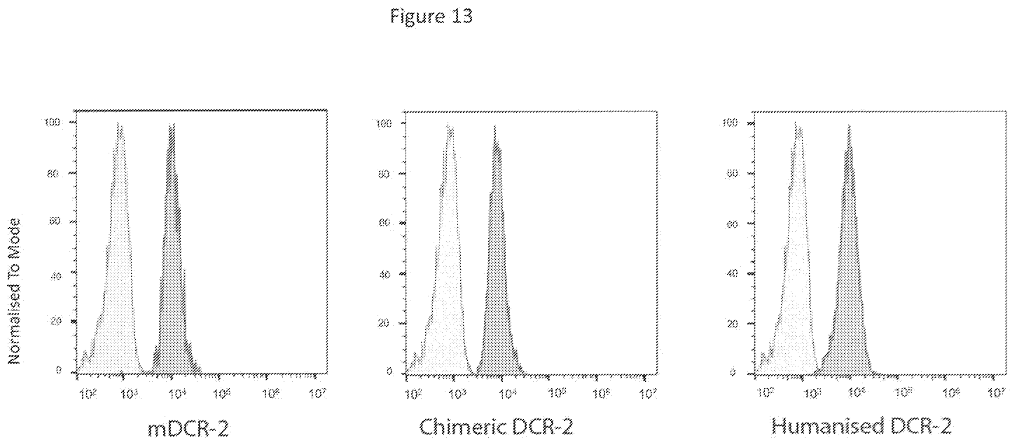

The amino acid sequence of the mouse VH and mouse VL of DCR-2 was aligned through an IgBLAST to human Ig sequences. The amino acids in the framework region of the VH and VL sequences were changed from mouse to human residues (underlined in FIGS. 11 and 12). No change to the CDR regions as these are most likely to be important in the binding to antigen. The amino acid sequence was codon optimised through the GENEART software for expression in Chinese hamster (Cricetulus griseus). The nucleic acid sequence encoding VH spliced to human IgG1 heavy chain was inserted into pcDNA3 and VL spliced to human kappa sequence was inserted into pcDNA3 and transfected into EXPI CHO for expression. Immunoglobulin was purified from the tissue culture supernatant by protein A affinity chromatography. The amino acid sequence of the humanized variable light chain is shown compared to mouse in FIG. 11, and the amino acid sequence of the humanized variable heavy chain is shown compared to mouse in FIG. 12. The purified antibody was assessed by bioanalyser for integrity and concentration and by flow cytometry for ability to bind cell surface CD300f. The results of its comparison of binding ability to mouse DCR-2 and chimeric DCR-2 is shown in FIG. 13.

In summary:
CD300f internalizes on cell lines and Mo-DC.
It is expressed on blood DC and monocytes with limited expression on other tissue.
Using DCR-2 to antigen load DC can produce an antigen specific T cell response
Crosslinking with DCR-2 leads to increase in activation markers on DC but not on monocytes
Using an allogeneic MLR there is a suggestion that myeloid DC crosslinked with DCR-2 are more stimulatory.
Crosslinking with DCR-2 leads to increased DC migration

SEQUENCES

DCR-2 VH region (SEQ ID NO: 1):
Met Glu Ser Gly Gly Gly Leu Val Gln Pro Gly Gly Pro Leu Lys Leu Ser Cys
Ala Ala Ser Gly Phe Gly Phe Ser Gly Ser Trp Met Ser Trp Val Arg Gln Ala
Pro Gly Lys Gly Leu Glu Trp Ile Gly Gln Ile Asn Pro Asp Ser Ser The Ile
Asn Tyr Thr Pro Ser Leu Lys Asp Lys Phe Ile Ile Ser Arg Asp Asn Ala Lys
Asn Thr Leu Tyr Leu Gln Ile Asn Lys Val Arg Ser Glu Asp Thr Ala Leu Tyr
Tyr Cys Ala Arg Arg Gly Phe Phe Glu Gly Tyr Ser Ala Trp Phe Ala Tyr Trp
CDR1 of VH of DCR-2 (SEQ ID NO: 2):
Gly Phe Gly Phe Ser Gly Ser Trp
CDR2 of VH of DCR-2 (SEQ ID NO: 3):
Ile Asn Pro Asp Ser Ser Thr Ile
CDR3 of VH of DCR-2 (SEQ ID NO: 4):
Ala Arg Arg Gly Phe Phe Glu Gly Tyr Ser Ala Trp Phe Ala Tyr
VL of DCR-2 (SEQ ID NO: 5):
Ile Leu Met Thr Gln Thr Pro Lys Phe Leu Leu Val Ser Ala Gly Asp Arg Val
Thr Ile Thr Cys Lys Ala Ser Gln Ser Val Ser Asn Asp Val Ala Trp Tyr Gln
Gln Lys Pro Gly Gln Ser Pro Ser Leu Leu Ile Tyr Tyr Ala Ser Asn Arg Asn
Thr Gly Val Pro Asp Arg Phe Thr Gly Ser Gly Tyr Glu Thr Asp Phe Thr Phe
Thr Ile Ser Thr Val Gln Ala Glu Asp Leu Ala Val Tyr Phe Cys Gln Gln Asp
Tyr Thr Ser Pro Trp Thr Phe Gly Gly Gly
CDR1 of VL of DCR-2 (SEQ ID NO: 6):
Gln Ser Val Ser Asn Asp
CDR2 of VL of DCR-2 (SEQ ID NO: 7):
Tyr Ala Ser
CDR3 of VL of DCR-2 (SEQ ID NO: 8):
Gln Gln Asp Tyr Thr Ser Pro Trp Thr
VH region of DCR-2 (SEQ ID NO: 9):
atggagtctg gaggtggcct ggtgcagcct ggaggacccc tgaaactctc ctgtgcagcc 60
tcaggattcg gttttagtgg atcttggatg agttgggtcc ggcaggctcc agggaaaggg 120
ctagaatgga ttggacaaat taatccagat agcagtacga taaattatac accatctcta 180
aaggataaat tcatcatctc cagagacaac gccaaaaata ccctgtacct gcaaattaac 240
aaagtgagat ctgaggacac agccctttat tactgtgcaa gacgggggtt ctttgaaggt 300
tactccgcct ggtttgctta ctgg 324
VL region of DCR-2 (SEQ ID NO: 10):
attttgatga cccagactcc caaattcctg cttgtatcag caggagacag ggtgaccata 60
acctgcaagg ccagtcagag tgtgagtaat gatgtagctt ggtaccaaca gaagccaggg 120
cagtctcctt cactcctgat atactatgca tccaatcgca acactggagt ccctgatcgc 180
ttcactggca gtggatatga gacggatttc actttcacca tcagcactgt gcaggctgaa 240
gacctggcag tttatttctg tcagcaggat tatacctctc cgtggacgtt cggtggaggc 300
Codon optimised chimeric heavy chain of DCR-2 (SEQ ID NO: 11):
atggtcctga gcctgctgta cctgctgaca gctctgcctg gcatcctgtc tgaggtccag 60
ctgcaagagt ctggccccat ggaatctggc ggaggattgg ttcaacctgg cggccctctg 120
aagctgtctt gtgccgcttc tggcttcggc ttctccggct cttggatgtc ctgggtccga 180
caggctcctg gcaaaggcct ggaatggatc ggccagatca accccgactc ctccaccatc 240
aactacaccc ctagcctgaa ggacaagttc atcatctccc gggacaacgc caagaacacc 300
ctgtacttgc agatcaacaa agtgcggagc gaggacaccg ctctgtacta ctgtgccaga 360
cggggcttct tcgagggcta ctctgcttgg tttgcctact ggggccaggg cacactggtc 420
acagtttctg ccgcctctac caagggaccc agcgttttcc ctctggctcc atcctccaag 480
tctacctctg gcggaacagc tgctctgggc tgcctggtca aggactactt tcctgagcca 540
gtgaccgtgt cctggaactc tggcgctctg acatctggcg tgcacacctt tccagctgtg 600
ctgcagtcct ccggcctgta ctctctgtcc tctgtcgtga ccgtgccttc cagctctctg 660
ggaacccaga cctacatctg caatgtgaac cacaagcctt ccaacaccaa ggtggacaag 720
aaggtggaac ccaagtcctg cgacaagacc cacacctgtc ctccatgtcc tgctccagaa 780
ctgctcggcg gaccttccgt gttcctgttt cctccaaagc ctaaggacac cctgatgatc 840
tctcggaccc ctgaagtgac ctgcgtggtg gtggatgtgt ctcacgagga tcccgaagtg 900
aagttcaatt ggtacgtgga cggcgtggaa gtgcacaatg ccaagaccaa gcctagagag 960
gaacagtaca actccaccta tagagtggtg tccgtgctga ccgtgctgca ccaggattgg 1020
ctgaacggca aagagtacaa gtgcaaggtg tccaacaagg ccctgcctgc tcctatcgaa 1080
aagaccatct ccaaggccaa gggccagcct agggaacccc aggtttacac cttgcctcca 1140
tctcgggacg agctgaccaa gaaccaggtg tccctgacct gtctcgtgaa gggcttctac 1200
ccctccgata tcgccgtgga atgggagtct aatggccagc ctgagaacaa ctacaagaca 1260
acccctcctg tgctggactc cgacggctca ttcttcctgt actccaagct gacagtggac 1320
aagtccagat ggcagcaggg caacgtgttc tcctgctccg tgatgcacga ggccctgcac 1380
aatcactaca cccagaagtc cctgtctctg tcccctggca ag 1420
Codon optimised chimeric light chain (SEQ ID NO: 12):
atggactctc aggcccaggt gctgatgctg ctgctgttgt gggtgtccgg cacctgtggc 60
gacatcctga tgacccagac tcctaagttc ctgctggtgt ctgccggcga cagagtgacc 120
atcacatgca aggcctctca gtccgtgtcc aacgacgtgg cctggtatca gcagaagcct 180
ggccagtctc ctagcctgct gatctactac gcctccaaca gaaacaccgg cgtgcccgat 240
agattcaccg gctctggcta cgagacagac ttcaccttca ccatctccac cgtgcaggcc 300
gaggatctgg ccgtgtactt ctgccagcaa gactacacct ctccatggac ctttggcgga 360
ggcaccaagc tggaaatcaa gcggacagtg gccgctcctt ccgtgttcat cttcccacct 420
tccgacgagc agctgaagtc tggcacagcc tctgtcgtgt gcctgctgaa caacttctac 480
cctcgggaag ccaaggtgca gtggaaggtg gacaatgccc tgcagtccgg caactcccaa 540
gagtctgtga ccgagcagga ctccaaggac agcacctaca gcctgtcctc cacactgacc 600
ctgtccaagg ccgactacga gaagcacaag gtgtacgcct gcgaagtgac ccatcagggc 660
ctgtctagcc ctgtgaccaa gtctttcaac cggggcgagt gc 720
Heavy chain encoded by optimised nucleotide sequence (SEQ ID NO: 13):
Met Val Leu Ser Leu Leu Tyr Leu Leu Thr Ala Leu Pro Gly Ile Leu Ser Glu
Val Gln Leu Gln Glu Ser Gly Pro Met Glu Ser Gly Gly Gly Leu Val Gln Pro
Gly Gly Pro Leu Lys Leu Ser Cys Ala Ala Ser Gly Phe Gly Phe Ser Gly Ser
Trp Met Ser Trp Val Arg Gln Ala Pro Gly Lys Gly Leu Glu Trp Ile Gly Gln
Ile Asn Pro Asp Ser Ser Thr Ile Asn Tyr Thr Pro Ser Leu Lys Asp Lys Phe
Ile Ile Ser Arg Asp Asn Ala Lys Asn Thr Leu Tyr Leu Gln Ile Asn Lys Val
Arg Ser Glu Asp Thr Ala Leu Tyr Tyr Cys Ala Arg Arg Gly Phe Phe Glu Gly
Tyr Ser Ala Trp Phe Ala Tyr Trp Gly Gln Gly Thr Leu Val Thr Val Ser Ala
Ala Ser Thr Lys Gly Pro Ser Val Phe Pro Leu Ala Pro Ser Ser Lys Ser Thr
Ser Gly Gly Thr Ala Ala Leu Gly Cys Leu Val Lys Asp Tyr Phe Pro Glu Pro
Val Thr Val Ser Trp Asn Ser Gly Ala Leu Thr Ser Gly Val His Thr Phe Pro
Ala Val Leu Gln Ser Ser Gly Leu Tyr Ser Leu Ser Ser Val Val Thr Val Pro
Ser Ser Ser Leu Gly Thr Gln Thr Tyr Ile Vys Asn Val Asn His Lys Pro Ser
Asn Thr Lys Val Asp Lys Lys Val Glu Pro Lys Ser Cys Asp Lys Thr His Thr
Cys Pro Pro Cys Pro Ala Pro Glu Leu Leu Gly Gly Pro Ser Val Phe Leu Phe
Pro Pro Lys Pro Lys Asp Thr Leu Met Ile Ser Arg Thr Pro Glu Val Thr Cys
Val Val Val Asp Val Ser His Glu Asp Pro Glu Val Lys Phe Asn Trp Tyr Val
Asp Gly Val Glu Val His Asn Ala Lys Thr Lys Pro Arg Glu Glu Gln Tyr Asn
Ser Thr Tyr Arg Val Val Ser Val leu Thr Val Leu His Gln Asp Trp Leu Asn
Gly Lys Glu Tyr Lys Cys Lys Val Ser Asn Lys Ala Leu Pro Ala Pro Ile Glu
Lys Thr Ile Ser Lys Ala Lys Gly Gln Pro Arg Glu Pro Gln Val Tyr Thr Leu
Pro Pro Ser Arg Asp Glu Leu Thr Lys Asn Gln Val Ser Leu Thr Cys Leu Val
Lys Gly Phe Tyr Pro Ser Asp Ile Ala Val Glu Trp Glu Ser Asn Glu Gln Pro
Glu Asn Asn Tyr Lys Thr Thr Pro Pro Val Leu Asp Ser Asp Gly Ser Phe Phe
Leu Tyr Ser Lys leu Thr Val Asp Lys ser Arg Trp Gln Gln Gly Asn Val Phe
Ser Cys Ser Val Met His Glu Ala Leu His Asn His Tyr Thr Gln Lys Ser Leu
Ser Leu Ser Pro Gly Lys
Light chain encoded by codon optimised nucleotide sequence
(SEQ ID NO: 14):
Met Asp Ser Gln Ala Gln Val Leu Met Leu Leu Leu Leu Trp Val Ser Gly Thr
Cys Gly Asp Ile Leu Met Thr Gln Thr Pro Lys Phe Leu Leu Val Ser Ala Gly
Asp Arg Val Thr Ile Thr Cys Lys Ala Ser Gln Ser Val Ser Asn Asp Val Ala
Trp Tyr Gln Gln Lys Pro Gly Gln Ser Pro Ser Leu Leu Ile Tyr Tyr Ala Ser
Asn Arg Asn Thr Gly Val Pro Asp Arg Phe Thr Gly Ser Gly Tyr Glu Thr Asp
Phe Thr Phe Thr Ile Ser Thr Val Gln Ala Glu Asp Leu Ala Val Tyr Phe Cys
Gln Gln Asp Tyr Thr Ser Pro Trp Thr Phe Gly Gly Gly Thr Lys Leu Glu Ile
Lys Arg Thr Val Ala Ala Pro Ser Val Phe Ile Phe Pro Pro Ser Asp Glu Gln
Leu Lys Ser Gly Thr Ala Ser Val Val Cys Leu Leu Asn Asn Phe Tyr Pro Arg
Glu Ala Lys Val Gln Trp Lys Val Asp Asn Ala Leu Gln Ser Gly Asn Ser Gln
Glu Ser Val Thr Glu Gln Asp Ser Lys Asp Ser Thr Tyr Ser Leu Ser Ser Thr
Leu Thr Leu Ser Lys Ala Asp Tyr Glu Lys His Lys Val Tyr Ala Cys Glu Val
Thr His Gln Gly Leu Ser Ser Pro Val Thr Lys Ser Phe Asn Arg Gly Glu Cys
Humanized VH of DCR-2 (SEQ ID NO: 15):
Met Glu Ser Gly Gly Gly Leu Val Gln Pro Gly Gly Ser Leu Arg Leu Ser Cys
Ala Ala Ser Gly Phe Gly Phe Ser Gly Ser Trp Met Ser Trp Val Arg Gln Ala
Pro Gly Lys Gly Leu Glu Trp Val Ala Asn Ile Asn Pro Asp Ser Ser Thr Ile
Tyr Tyr Val Asp Ser Val Lys Gly Arg Phe The Ile Ser Arg Asp Asn Ala Lys
Asn Ser Leu Tyr Leu Gln Met Asn Ser Leu Arg Ala Glu Asp Thr Ala Val Tyr
Tyr Cys Ala Arg Arg Gly Phe Phe Glu Gly Tyr Ser Ala Trp Phe Ala Tyr Trp
Humanized VL of DC-2 (SEQ ID NO: 16):
Ile Gln Met Thr Gln Ser Pro Ser Ser Leu Ser Ala Ser Val Gly Asp Arg Val
Thr Ile Thr Cys Lys Ala Ser Gln Ser Val Ser Asn Asp Leu Asn Trp Tyr Gln
Gln Lys Pro Gly Lys Ala Pro Lys Leu Leu Ile Tyr Tyr Ala Ser Asn Leu Glu
Thr Gly Val Pro Ser Arg Phe Ser Gly Ser Gly Ser Gly Thr Asp Phe Thr Phe
Thr Ile Ser Ser Leu Gln Pro Glu Asp Ile Ala Thr Tyr Tyr Cys Gln Gln Asp
Tyr Thr Ser Pro Trp Thr Phe Gly Gly Gly
Heavy chain joining region (SEQ ID NO: 17):
Gly Gln Gly Thr Leu Val Thr Val

1-18. (canceled)

19. A method of promoting or increasing a T cell response to a target antigen in a subject in need thereof, the method comprising administering to the subject an effective amount of a CD300f binding protein and the target antigen.

20. The method of claim 19, wherein the promoting or increasing the T cell response to the target antigen treats a disease or condition requiring a T cell response to the target antigen.

21. The method of claim 19, wherein the CD300f binding protein is coupled to the target antigen.

22. The method of claim 1, wherein the target antigen is a cancer antigen or a viral antigen.

23. (canceled)

24. A composition for antigen loading an antigen presenting cell or precursor thereof with a target antigen, the composition comprising a CD300f binding protein and the target antigen.

25. The composition of claim 24, wherein the composition is a pharmaceutical composition.

26. An immunoconjugate for antigen loading an antigen presenting cell or precursor thereof with a target antigen, the immunoconjugate comprising a CD300f binding protein coupled to the target antigen.

27. The immunoconjugate of claim 26, wherein the target antigen is a cancer antigen or a viral antigen.

28. (canceled)

29. A method of producing an antigen-loaded antigen presenting cell or precursor thereof which is capable of presenting a target antigen to a T cell, the method comprising contacting an antigen presenting cell or precursor thereof with a CD300f binding protein in the presence of the target antigen.

30-34. (canceled)

35. The method of claim 19, wherein the CD300f binding protein comprises:
(a) a heavy chain variable region which comprises a CDR1 comprising the amino acid sequence represented by SEQ ID NO: 2, a CDR2 comprising the amino acid sequence represented by SEQ ID NO:3, and a CDR3 comprising the amino acid sequence represented by SEQ ID NO: 4; and
(b) a light chain variable region which comprises a CDR1 comprising the amino acid sequence represented by SEQ ID NO: 6, a CDR2 comprising the amino acid sequence represented by SEQ ID NO:7, and a CDR3 comprising the amino acid sequence represented by SEQ ID NO: 8.

36. The method of claim 35, wherein the heavy chain variable region is at least 70% identical to the amino acid sequence of SEQ ID NO: 1 or 15, and the light chain variable region is at least 70% identical to SEQ ID NO: 5 or 16.

37. The immunoconjugate of claim 26, wherein the CD300f binding protein comprises:
(a) a heavy chain variable region which comprises a CDR1 comprising the amino acid sequence represented by SEQ ID NO: 2, a CDR2 comprising the amino acid sequence represented by SEQ ID NO:3, and a CDR3 comprising the amino acid sequence represented by SEQ ID NO: 4; and
(b) a light chain variable region which comprises a CDR1 comprising the amino acid sequence represented by SEQ ID NO: 6, a CDR2 comprising the amino acid sequence represented by SEQ ID NO:7, and a CDR3 comprising the amino acid sequence represented by SEQ ID NO: 8.

38. The immunoconjugate of claim 37, wherein the heavy chain variable region is at least 70% identical to the amino acid sequence of SEQ ID NO: 1 or 15, and the light chain variable region is at least 70% identical to SEQ ID NO: 5 or 16.

39. The composition of claim 24, wherein the CD300f binding protein comprises:
(a) a heavy chain variable region which comprises a CDR1 comprising the amino acid sequence represented by SEQ ID NO: 2, a CDR2 comprising the amino acid sequence represented by SEQ ID NO:3, and a CDR3 comprising the amino acid sequence represented by SEQ ID NO: 4; and
(b) a light chain variable region which comprises a CDR1 comprising the amino acid sequence represented by SEQ ID NO: 6, a CDR2 comprising the amino acid sequence represented by SEQ ID NO:7, and a CDR3 comprising the amino acid sequence represented by SEQ ID NO: 8.

40. The method of claim 29, wherein the CD300f binding protein comprises:
(a) a heavy chain variable region which comprises a CDR1 comprising the amino acid sequence represented by SEQ ID NO: 2, a CDR2 comprising the amino acid sequence represented by SEQ ID NO:3, and a CDR3 comprising the amino acid sequence represented by SEQ ID NO: 4; and
(b) a light chain variable region which comprises a CDR1 comprising the amino acid sequence represented by SEQ ID NO: 6, a CDR2 comprising the amino acid sequence represented by SEQ ID NO:7, and a CDR3 comprising the amino acid sequence represented by SEQ ID NO: 8.

41. (canceled)

42. The method of claim 40, wherein the heavy chain variable region is at least 70% identical to the amino acid sequence of SEQ ID NO: 1 or 15, and the light chain variable region is at least 70% identical to SEQ ID NO: 5 or 16.

43. The composition of claim 39, wherein the heavy chain variable region is at least 70% identical to the amino acid sequence of SEQ ID NO: 1 or 15, and the light chain variable region is at least 70% identical to SEQ ID NO: 5 or 16.

44. The method of claim 29, wherein contacting of the antigen presenting cell or precursor thereof with the CD300f binding protein promotes activation of the antigen presenting cell or precursor thereof.

45. The method of claim 29, wherein the antigen presenting cell or precursor thereof is a dendritic cell or a monocyte.

46. The method of claim 45, wherein the dendritic cell is a myeloid dendritic cell.

47. The method of claim 29, wherein the CD300f binding protein is coupled to the antigen.

48. The method of claim 29, wherein activation of the antigen presenting cell or precursor thereof increases expression of one or more activation markers.

49. The method of claim 48, wherein the one or more activation markers is one or more proteins selected from CD80, CD83, CD86, and HLA-DR.

50. The method of claim 49, wherein the antigen presenting cell is a dendritic cell and the one or more activation markers are CD80, CD83 and CD86.

51. The method of claim 24, wherein the CD300f binding protein is coupled to the antigen.


SEQUENCE LISTING (17 sequences)

SEQ ID NO: 1 - protein, 108 aa, Artificial Sequence
  DCR-2 VH region
MESGGGLVQPGGPLKLSCAASGFGFSGSWMSWVRQAPGKGLEWIGQINPDSSTINYTPSLKDKFIISRDNAKNTLYLQINKVRSEDTALYYCARRGFFEGYSAWFAYW

SEQ ID NO: 2 - protein, 8 aa, Artificial Sequence
  CDR1 of VH of DCR-2
GFGFSGSW

SEQ ID NO: 3 - protein, 8 aa, Artificial Sequence
  CDR2 of VH of DCR-2
INPDSSTI

SEQ ID NO: 4 - protein, 15 aa, Artificial Sequence
  CDR3 of VH of DCR-2
ARRGFFEGYSAWFAY

SEQ ID NO: 5 - protein, 100 aa, Artificial Sequence
  VL of DCR-2
ILMTQTPKFLLVSAGDRVTITCKASQSVSNDVAWYQQKPGQSPSLLIYYASNRNTGVPDRFTGSGYETDFTFTISTVQAEDLAVYFCQQDYTSPWTFGGG

SEQ ID NO: 6 - protein, 6 aa, Artificial Sequence
  CDR1 of VL of DCR-2
QSVSND

SEQ ID NO: 7 - protein, 3 aa, Artificial Sequence
  CDR2 of VL of DCR-2
YAS

SEQ ID NO: 8 - protein, 9 aa, Artificial Sequence
  CDR3 of VL of DCR-2
QQDYTSPWT

SEQ ID NO: 9 - DNA, 324 bp, Artificial Sequence
  VH region of DCR-2
atggagtctggaggtggcctggtgcagcctggaggacccctgaaactctcctgtgcagcctcaggattcggttttagtggatcttggatgagttgggtccggcaggctccagggaaagggctagaatggattggacaaattaatccagatagcagtacgataaattatacaccatctctaaaggataaattcatcatctccagagacaacgccaaaaataccctgtacctgcaaattaacaaagtgagatctgaggacacagccctttattactgtgcaagacgggggttctttgaaggttactccgcctggtttgcttactgg

SEQ ID NO: 10 - DNA, 300 bp, Artificial Sequence
  VL region of DCR-2
attttgatgacccagactcccaaattcctgcttgtatcagcaggagacagggtgaccataacctgcaaggccagtcagagtgtgagtaatgatgtagcttggtaccaacagaagccagggcagtctccttcactcctgatatactatgcatccaatcgcaacactggagtccctgatcgcttcactggcagtggatatgagacggatttcactttcaccatcagcactgtgcaggctgaagacctggcagtttatttctgtcagcaggattatacctctccgtggacgttcggtggaggc

SEQ ID NO: 11 - DNA, 1422 bp, Artificial Sequence
  codon optimised chimeric heavy chain
atggtcctgagcctgctgtacctgctgacagctctgcctggcatcctgtctgaggtccagctgcaagagtctggccccatggaatctggcggaggattggttcaacctggcggccctctgaagctgtcttgtgccgcttctggcttcggcttctccggctcttggatgtcctgggtccgacaggctcctggcaaaggcctggaatggatcggccagatcaaccccgactcctccaccatcaactacacccctagcctgaaggacaagttcatcatctcccgggacaacgccaagaacaccctgtacttgcagatcaacaaagtgcggagcgaggacaccgctctgtactactgtgccagacggggcttcttcgagggctactctgcttggtttgcctactggggccagggcacactggtcacagtttctgccgcctctaccaagggacccagcgttttccctctggctccatcctccaagtctacctctggcggaacagctgctctgggctgcctggtcaaggactactttcctgagccagtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaatgccaagaccaagcctagagaggaacagtacaactccacctatagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgatatcgccgtggaatgggagtctaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggcaag

SEQ ID NO: 12 - DNA, 702 bp, Artificial Sequence
  codon optimised chimeric light chain
atggactctcaggcccaggtgctgatgctgctgctgttgtgggtgtccggcacctgtggcgacatcctgatgacccagactcctaagttcctgctggtgtctgccggcgacagagtgaccatcacatgcaaggcctctcagtccgtgtccaacgacgtggcctggtatcagcagaagcctggccagtctcctagcctgctgatctactacgcctccaacagaaacaccggcgtgcccgatagattcaccggctctggctacgagacagacttcaccttcaccatctccaccgtgcaggccgaggatctggccgtgtacttctgccagcaagactacacctctccatggacctttggcggaggcaccaagctggaaatcaagcggacagtggccgctccttccgtgttcatcttcccaccttccgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgc

SEQ ID NO: 13 - protein, 474 aa, Artificial Sequence
  heavy chain encoded by codon optimised nucleotide sequence
MVLSLLYLLTALPGILSEVQLQESGPMESGGGLVQPGGPLKLSCAASGFGFSGSWMSWVRQAPGKGLEWIGQINPDSSTINYTPSLKDKFIISRDNAKNTLYLQINKVRSEDTALYYCARRGFFEGYSAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 234 aa, Artificial Sequence
  light chain encoded by codon optimised nucleotide sequence
MDSQAQVLMLLLLWVSGTCGDILMTQTPKFLLVSAGDRVTITCKASQSVSNDVAWYQQKPGQSPSLLIYYASNRNTGVPDRFTGSGYETDFTFTISTVQAEDLAVYFCQQDYTSPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 108 aa, Artificial Sequence
  humanised VH of DCR-2
MESGGGLVQPGGSLRLSCAASGFGFSGSWMSWVRQAPGKGLEWVANINPDSSTIYYVDSVKGRFTISRDNAKNSLYLQMNSLRAEDTAVYYCARRGFFEGYSAWFAYW

SEQ ID NO: 16 - protein, 100 aa, Artificial Sequence
  humanised light chain variable region of DCR-2
IQMTQSPSSLSASVGDRVTITCKASQSVSNDLNWYQQKPGKAPKLLIYYASNLETGVPSRFSGSGSGTDFTFTISSLQPEDIATYYCQQDYTSPWTFGGG

SEQ ID NO: 17 - protein, 8 aa, Artificial Sequence
  DCR-2 heavy joining region
GQGTLVTV